(19) United States
(12) Patent Application Publication
(10) Pub. No.: US 2015/0037251 A1
(43) Pub. Date: Feb. 5, 2015

(54) MAMMALIAN HOMOLOGUES OF FLOWER, THEIR USE IN CANCER DIAGNOSTICS, PREVENTION AND TREATMENT

(71) Applicants: CENTRO NACIONAL DE INVESTIGACIONES ONCOLOGICAS, Madrid (ES); UNIVERSITAT BERN, Bern (CH)

(72) Inventors: Christa Rhiner, Biel (CH); Evgeniya Pertova, Paris (FR); Jesus M. Lopez-Gay, Paris (FR); Eduardo Moreno, Biel (CH)

(73) Assignees: UNIVERSITAT BERN, Bern (CH); CENTRO NACIONAL DE INVESTIGACIONES ONCOLOGICAS, Madrid (ES)

(21) Appl. No.: 14/379,296

(22) PCT Filed: Feb. 18, 2013
(86) PCT No.: PCT/EP2013/053163

§ 371 Date: Aug. 17, 2014

(30) Foreign Application Priority Data
Feb. 17, 2012 (EP) 12155942.1
Feb. 20, 2012 (EP) 12156256.5

(51) Int. Cl. C12Q 1/68; C07K 16/30; G01N 33/574

(52) U.S. Cl. CPC C12Q 1/6886; G01N 33/57492; C07K 16/30; C12Q 2600/158; C12Q 2600/112; G01N 2333/705
USPC 424/1.49; 435/6.14; 435/6.12; 435/6.11; 435/7.93; 530/391.3; 530/387.9; 530/387.3; 536/24.31; 424/139.1; 424/133.1; 424/183.1; 514/44 A; 435/320.1

(57) ABSTRACT

The invention relates inhibiting nucleic acids directed at mammalian homologues of the Drosophila fwe gene (Flower) and to antibodies against the respective proteins, and their use in diagnosing, preventing and treating cancer.

The present invention relates to mammalian homologues of the drosophila Fwe (Flower) protein and its encoding nucleic acids. The invention provides means and methods, particularly antibodies and inhibitory nucleic acids useful for diagnostics, prevention and treatment of cancer.

Tumor formation is preceded by clonal expansion of pretumoral, mutant cells. Clones of pretumoral cells are often invisible to the naked eye, due to absence of morphological alterations in the tissue. It was proposed that such clones facilitate their own expansion by interacting with the surrounding normal cells (Moreno (2008), Nat Rev Cancer 8, 141-147). Such interaction can be based on the relative cellular fitness status of the cell: cells of higher fitness are selected and persist in the tissue at the expense of less fit ones.

Recent studies demonstrate that, in Drosophila, the mechanism through which cells of lower fitness are recognized and eliminated from a tissue depends on the function of an extracellular molecular code, called “The Flower Code” (Rhiner et al. (2010) Dev. Cell 18, 1-14). This code is based on three isoforms of the cell membrane protein Flower (Fwe): Few(ubi), Few(Lose-A) and Few(Lose-B). Basal levels of Fweubi are constantly produced in the Drosophila wing imaginal disc, but when cells of lower relative fitness (but which are viable on their own) appear, they are recognized due to the up-regulation of the few(Lose) isoforms, which eventually leads to caspase 3 activation in such “loser” cells.

Agents that regulate cell competition in mammals have not been described in the prior art. The mammalian homologues of Drosophila fwe (dfwe) have not been studied so far and their function is not known. The single mouse C9orf7 gene has been isolated and described (Yao et al. (2008) Cell 138, 947-960), but its role in cell competition has not been described nor suggested previously. The longest C9orf7 protein isoform shares just 30% identity with the longest Drosophila Fwe isoform (Few(ubi)).

The present invention improves on the state of the art by identifying mammalian, particularly human, homologues of Fwe, thereby providing means and methods for diagnosis, prevention and therapy of cancer, and furthermore facilitating assay systems for identifying cancer drug development candidates.

dfwe gene has a single predicted homologue in mice: 5930434B04Rik (Accession number: MGI:1924317). The mouse C9orf7 gene occupies 11.2 kb on chromosome 2 (qA3). This gene shares 92% identity with the longest protein isoform of its human orthologue. Mouse Flower (mFwe) encodes six different transcripts, normally expressed at low levels in adult tissues, which are translated into four protein isoforms.

By expressing individual mouse C9orf7 isoforms in Drosophila to test their function in established competition assays, the present inventors have unexpectedly found that certain mammalian isoforms of Flower behave as Loser forms, establishing their role in cell competition mechanisms. Absence of the C9orf7 gene in mice results in normal development, morphology and growth, but C9orf7 knock-out mice show a very significant protection against skin carcinogenesis. Upon treatment with 7,12-dimethylbenz[a]anthracene (DMBA) and 12-O-tetradecanoylphorbol-13-acetate (TPA), C9orf7 knock-out mice show 60% reduction in the number of papillomas. 20% of the mice remain papilloma free after the standard 15 weeks treatment. In addition, the few papillomas that appear in the C9orf7 knock-out mice are less proliferative.

In other words, elimination of the C9orf7 gene specifically impairs cell competition driven malignant growth, but not normal growth, implying that certain pre-cancerous cells can misuse the C9orf7 code in order to proliferate and contribute to tumor formation.

In mice, C9orf7 isoforms 1 and 3 behave as Loser isoforms and some of these isoforms, especially C9orf7 1, are up-regulated in the tissue that surrounds a papilloma. It is inferred from the data presented herein that DMBA/TPA-induced skin papillomas—and by extension, naturally occurring carcinomas in mammals—use mammalian homologues of dFwe to grow at the expense of the surrounding normal skin.

Furthermore, the data presented herein suggest the utility of abrogating Fwe signalling either on a protein or nucleic acid level in order to deprive nascent tumors or pre-cancerous lesions of what appears to be an important mechanism of propagation. The importance of Fwe expression in cancer may well not be limited to early stage tumor formation. Recent results describe the up-regulation of competition related genes also for established tumors and implicate this mechanism in metastasis (Petrova et al., Communicative & Integrative Biology (2011) 4, 1-4).

A “pre-cancerous state” or a “pre-cancerous lesion” in the context of the present specification refers to tissue comprising a cell population having undergone de-differentiation, dysplasia, or any other detectable change of healthy tissue towards neoplasia, particularly malignant neoplasia (cancer).

Human homologue of Fwe sequence data:

SEQ ID NO 1 is Ensembl transcript ID ENST00000316948.

SEQ ID NO 2 is Ensembl transcript ID ENST00000291722.

SEQ ID NO 3 is Ensembl transcript ID ENST00000444798.

SEQ ID NO 4 is Ensembl transcript ID ENST00000535514.

SEQ ID NO 5 is Ensembl transcript ID ENST00000540581.

SEQ ID NO 6 is Ensembl transcript ID ENST00000542192.

SEQ ID NO 7 is Ensembl protein ID ENSP00000317121.

SEQ ID NO 8 is Ensembl protein ID ENSP00000291722.

SEQ ID NO 9 is Ensembl protein ID ENSP00000414495.

SEQ ID NO 10 is Ensembl protein ID ENSP00000444402.

SEQ ID NO 11 is Ensembl protein ID ENSP00000440832.

SEQ ID NO 12 is Ensembl protein ID ENSP00000444328.

Sequences of SEQ ID NO 1 to 6 correspond to the coding nucleic acid sequences encoding the six human Flower homologues. Sequences 007 to 012 correspond to the corresponding encoded proteins, respectively.

SEQ ID NO 13 corresponds to mRNA of C9orf7-202_ENST00000540581, from the ATG in 1st exon to the final nucleotide of its 3th exon (SEQ ID NO-5) RNAi target sequence. SEQ ID 013 has no known off-targets (no other 19-nucleotides sequences found similar to this in the human genome) and targets all coding isoforms of human Fwe.

SEQ ID NO 14 is an C-terminal extracellular loop comprised in SEQ ID NO 7, 8 or 10.

SEQ ID NO 15 is an extracellular loop between transmembrane domains 1 and 2.

SEQ ID NO 16 is an extracellular domain at the C-terminal of SEQ ID 11 and 12.

SEQ ID NO 17 is an extracellular loop between transmembranes 1 and 2 of SEQ ID 11 and 12.

According to a first aspect of the invention a method for diagnosing cancer, a tumor disease or a pre-cancerous state in a human subject is provided, comprising the steps of:
a) determining in a biological sample obtained from said human subject
   i. the presence, location, and/or quantity of a nucleic acid sequence identified by any one of SEQ ID NO 1, 2, 3, 4, 5, 6 or 13;
   ii. the presence, location, and/or quantity of a protein identified by any one of SEQ ID NO 7, 8, 9, 10, 11, 12, 14, 15, 16 and/or 17,
b) comparing said presence, location, and/or quantity of said nucleic acid or said protein to a standard.

According to an alternative of this first aspect of the invention, a method for assigning to a biological sample a diagnostic score value is provided, comprising:
a) determining
   i. the presence, location, and/or quantity of gene expression of a nucleic acid having at least 80%, 85%, 90%, 92%, 93%, 94%, 95%, 96%, 97%, 98% or 99% sequence identity to one of SEQ ID NO 1, 2, 3, 4, 5, 6 and/or 13 or
   ii. the presence, location, expression and/or concentration of a protein having at least 80%, 85%, 90%, 92%, 93%, 94%, 95%, 96%, 97%, 98% or 99% sequence identity to one of SEQ ID NO 7, 8, 9, 10, 11, 12, 14, 15, 16 and/or 17,
   in a biological sample obtained from a human subject,
b) comparing said gene expression level or said protein presence, location, expression and/or concentration to a standard, and
c) assigning the sample a diagnostic score value as a function of the result of step b).

In one embodiment, said diagnostic score value relates to a likelihood of said sample representing a tumor tissue, particularly a carcinoma tissue.

In some embodiments, the method is performed ex-vivo.

In some embodiments, said biological sample is selected from the group comprising a blood sample, a plasma sample, a serum sample, a saliva sample, a biopsy sample, a tumor sample and a tissue sample.

In one embodiment, the presence of a nucleic acid identified by SEQ ID NO 2 and/or 6 is determined. In one embodiment, the presence of a protein identified by SEQ ID NO 8 or 12 is determined (these sequences represent Fwe(Loser)).

In one embodiment, the presence of a nucleic acid identified by SEQ ID NO 1 and/or 5 is determined. In one embodiment, the presence of a protein identified by SEQ ID NO 7 or 11 is determined (these sequences represent Fwe(Ubi)).

In some embodiments, the presence, location, and/or quantity of a nucleic acid sequence is determined by RT-PCR (reverse transcriptase polymerase chain reaction) or FISH (fluorescence in-situ-hybridization). RT-PCR allows for the precise determination of transcript levels in the sample. In one embodiment, an RT-PCR primer-probe combination is selected that amplifies a consensus sequence common to all coding sequences (SEQ ID NO 1 to 6). In one embodiment, this consensus sequence is the sequence of SEQ ID NO 13.

In one embodiment, an RT-PCR primer-probe combination is selected that amplifies a sequence specific for SEQ ID NO 1. In one embodiment, an RT-PCR primer-probe combination is selected that amplifies a sequence specific for SEQ ID NO 2. In one embodiment, an RT-PCR primer-probe combination is selected that amplifies a sequence specific for SEQ ID NO 3. In one embodiment, an RT-PCR primer-probe combination is selected that amplifies a sequence specific for SEQ ID NO 4. In one embodiment, an RT-PCR primer-probe combination is selected that amplifies a sequence specific for SEQ ID NO 5. In one embodiment, an RT-PCR primer-probe combination is selected that amplifies a sequence specific for SEQ ID NO 6.

In one embodiment, a “multiplex” set-up is chosen wherein primers and probes are provided that facilitate concomitant and specific detection of two, three, four, five or six sequences side-by-side within the same sample. While a certain amount of work is necessary to devise primer-probe combinations, the level of experimentation to arrive at multiplex RT-PCR combinations is well within the skill of the skilled artisan. For example, US2011136178 (hereby incorporated by reference) provides modified primers facilitating the design of multiplex RT mixes.

In one embodiment, an RT-PCR primer-probe combination is selected that amplifies sequences specific for SEQ ID NO 1 and 5. In one embodiment, an RT-PCR primer-probe combination is selected that amplifies sequences specific for SEQ ID NO 2 and 6.

In one embodiment, FISH is used to determine the localisation and distribution of nucleic acid sequences encoding the human homologue of Flower in a tissue sample.

For protocols on FISH, see Müller et al. Cold Spring Harbor Protocols 2007 (doi:10.1101/pdb.prot4730); Raj et al. 2008, Nature Methods 5, 877-879 doi:10.1038/nmeth.1253, and references cited therein.

In an alternative of this aspect of the invention, the presence, location, and/or quantity of a protein identified by one of SEQ ID NO 7, 8, 9, 10, 11, 12, 14, 15, 16 and/or 17 (the human homologue of Flower protein sequences) is determined. Any of the methods known to the skilled artisan for determining the presence, location or quantity of a specific protein in a tissue sample or other biological specimen obtainable ex-vivo can be employed. A ligand raised against and/or specific for the target protein, having a high affinity for the target protein (typically characterized by a dissociation constant in the range of 10E-7 to 10E-9 mol/l or lower) is brought into contact with the sample under conditions allowing for the specific binding of the ligand to its target, the sample is washed and the presence of bound ligand, or its quantity, is determined. In some embodiments, the ligand is an antibody (particularly a monoclonal antibody), an antibody-fragment or an antibody-like molecule.

In some embodiments, binding is determined by measuring the signal of a label attached to the ligand. Examples of suitable labels are fluorescent molecules such as dye molecules or fluorescent proteins, radioactive labels such as a radioisotope, or enzymes capable of mediating a reaction that can be employed to quantify the presence of the ligand directly, such as by luminescence (e.g., luciferase) or conversion of a suitable substrate to a dye molecule (e.g., peroxidase), or by catalyzing the attachment of reporter molecules (e.g., SNAP-tag).

Fluorescently labeled antibodies are particularly suitable to practice the method of the invention, as far as detection of protein is the objective.

According to one embodiment, an antibody specific for an extracellularly exposed amino acid sequence of a protein identified by one of SEQ ID NO 7, 8, 9, 10, 11, 12, 14, 15, 16 and/or 17 is employed for determination of protein presence, location, and/or quantity.

In one embodiment, a ligand, particularly an antibody, specific for an extracellularly exposed amino acid sequence of SEQ ID NO 7 is employed. “Specific” in the context of this embodiment refers to the ability of this ligand to bind to the extracellular part of SEQ ID NO 7, but not to at least one of SEQ ID NO 8, 9, 10, 11 or 12, thus enabling discrimination of cells or tissues that express SEQ ID NO 7 from those that express SEQ ID NO 8, 9, 10, 11 or 12. In some embodiments, a ligand is employed that specifically binds to an extracellular part of only one of the proteins identified by SEQ ID NO 7, 8, 9, 10, 11 or 12, and to none of the others. Such ligand is referred to as “single isoform specific ligand”.

According to one embodiment, 2, 3, 4, 5 or 6 single isoform specific ligands specific for any combination of proteins SEQ ID NO 7, 8, 9, 10, 11 and 12 are employed.

In one embodiment, the disease to be diagnosed is a neoplastic disease. In one embodiment, the disease is a carcinoma (cancer of epithelial origin).

According to another alternative of this first aspect of the invention, the method is practiced in-vivo and the presence of tissue expressing a human homologue of Flower as specified by one of the sequences of SEQ ID NO 7, 8, 9, 10, 11, 12, 14, 15, 16 and/or 17 is detected by binding of a ligand to an extracellular amino acid sequence of any or all of SEQ ID NO 7, 8, 9, 10, 11, 12, 14, 15, 16 and/or 17, and binding is determined by detecting a label attached to said ligand. Among the suitable ligands for this in-vivo alternative of the diagnostic method of the invention are radiolabelled ligands, ligands labeled by near-infrared dye molecules, PET- or SPECT-tracer labeled ligands or ligands labeled by NMR contrast agents such as gadolinium atoms. In some embodiments, such ligand is an antibody.

According to a second aspect of the invention, a ligand capable of selectively binding to a protein identified by one of SEQ ID NO 7, 8, 9, 10, 11, 12, 14, 15, 16 and/or 17, is provided, wherein said ligand is covalently attached to a detectable label or wherein the ligand comprises a detectable label.

Alternatively, the ligand comprises the detectable label. In some embodiments, the detectable label is part of the ligand structure. In one embodiment, a radioisotope is comprised in an amino acid incorporated in a peptide chain forming said ligand.

In some embodiments, the label the detectable label is a radioisotope or dye molecule, and is attached to the ligand covalently or via a covalently attached chelator molecule such as DOTA (1,4,7,10-tetraazacyclododecane-1,4,7,10-tetraacetic acid), NOTA (1,4,7-triazacyclononane-N,N′,N″-triacetic acid), HYNIC (6-Hydrazinopyridine-3-carboxylic acid) or others. In some embodiments, a radioactive label is attached to the ligand as a nanoparticle.

According to one embodiment of this aspect of the invention, the detectable label is a radiolabel for PET (positron emission tomography) or SPECT (Single-photon emission computed tomography), such as one of the radioisotopes carbon-11, nitrogen-13, oxygen-15, fluorine-18, gallium-68, technetium-99m, indium-111 or iodine-123, iodine-124. The radiolabel may be comprised in the ligand by incorporation or covalent coupling to the peptide itself, or a carrier molecule attached thereto.

In some embodiments, the detectable label is a near infrared fluorescent dye. One example for such dye is an indotricarbocyanine dye. Near infrared dyes have been described, inter alia, by Umezawa (J. Am. Chem. Soc. (2008) 130, 1550-1551) and are available, for example, from Amersham/GE Healthcare (Little Chalfont, GB), mivenion GmbH (Berlin, DE), and LI-COR Biosciences (Lincoln, Nebr., USA).

In some embodiments, the ligand is an antibody, an antibody-fragment, an antibody-like molecule, or a nucleic acid aptamer.

Methods for generating antibodies against a protein identified by one of SEQ ID NO 7, 8, 9, 10, 11, 12, 14, 15, 16 and/or 17, particularly the extracellular part of SEQ ID NO 7, 8, 9, 10, 11 and 12, are known in the art. They include, for example, immunization of mice with such protein, or soluble parts thereof.

An antibody fragment may be the Fab domain of an antibody (the antigen binding region of an antibody) or a single chain antibody (scFv), which is a fusion protein consisting of the variable regions of light and heavy chains of an antibody connected by a peptide linker. An antibody-like molecule may also be a repeat protein, such as a designed ankyrin repeat protein (Zurich Univ., Switzerland and Molecular Partners AG, Zurich, Switzerland; see US20120142611 (A1), incorporated by reference herein). An antibody fragment or an antibody-like molecule may be manufactured by methods such as recombinant protein expression.

Suitable ligands for practicing the invention may also be developed by evolutive methods such as phage display, ribosome display or SELEX, wherein polypeptides or oligonucleotides (aptamers) are selected according to their binding affinity to a target of interest. Additionally, ligands of higher affinity may be identified by reiterative rounds of evolution and selection of the amino acid sequence or nucleotide sequence.

According to one embodiment of the invention, a ligand as set forth above is provided, which comprises or is covalently linked to a radioisotope emitting beta or gamma radiation. A radioisotope such as carbon-11 for example may form part of the peptide backbone, in embodiments where the ligand is a polypeptide, e.g. an antibody. Alternatively, a radioisotope is attached to the side chain of an amino acid constituting the ligand, such as may be, by way of non-limiting example, a tyrosine, phenylalanine or histidine having an iodine radioisotope attached to its aromatic ring.

According to an alternative to this second aspect of the invention, the ligand according to this second aspect of the invention, in general form or in any of the specified embodiments, is provided for use in a method for detecting cancer or a precancerous state in a patient.

In some embodiments, the patient is a human being. In some embodiments, the method is practiced ex-vivo. In some other embodiments, the method is practiced in-vivo.

According to a third aspect of the invention, a nucleic acid molecule specifically hybridizing to a nucleic acid sequence identified by one of SEQ ID NO 1, 2, 3, 4, 5, 6 or 13 is provided, said nucleic acid molecule being covalently attached to a detectable label.

In some embodiments of this aspect of the invention, the nucleic acid molecule comprises a fluorescent dye molecule, for example for use as a probe in real-time PCR methods such as RT-PCR or for use as a FISH probe.

According to an alternative to this third aspect of the invention, the nucleic acid molecule according to this third aspect of the invention is provided for use in a method for detecting cancer or a precancerous state in a patient.

In some embodiments, the patient is a human being. In some embodiments, the method is practiced ex-vivo. In some other embodiments, the method is practiced in-vivo.

According to a fourth aspect of the invention, a ligand capable of selectively binding to a protein identified by one of SEQ ID NO 7, 8, 9, 10, 11, 12, 14, 15, 16 and/or 17 is provided for use in a method for preventing or treating a disease, particularly cancer, more particularly carcinoma or a pre-cancerous state.

In some embodiments, the ligand is an antibody, an antibody-fragment, an antibody-like molecule, or a nucleic acid aptamer. In some embodiments, the ligand is a human or humanized immunoglobulin gamma or a fragment thereof. In some embodiments, the ligand is an antibody or a fragment of an antibody raised against a protein identified by one of SEQ ID NO 7, 8, 9, 10, 11, 12, 14, 15, 16 and/or 17.

In some embodiments embodiment of this fourth aspect of the invention, the ligand is covalently attached to a therapeutic radioisotope or a cancer drug or toxin. Non-limiting examples for therapeutic radioisotopes are phosphorus-32, strontium-89, ytrrium-90, iodine-125 and -131, samarium-153, erbium-169, lutetium-177 and rhenium-186/188. Non-limiting examples for toxins and cancer drugs are ricin toxin, diphtheria toxin, anthrax toxin, pro-aerolysin, pseudomonas exotoxin, shigella toxin, cone snail neurotoxin, auristatin, doxorubicin, daunorubicin, taxol, irinotecan, vincristine, vinblastine, cisplatin, carboplatin, oxaliplatin and ifosfamideetoposide.

Alternatively, the antibody is provided as is, i.e. without attachment. As the examples of the present description illustrate, abrogation of the biological function of mammalian homologues of Flower is sufficient to prevent cancer from developing under certain circumstances, or for certain types of tumor.

The binding of ligand results in blocking the role of the Flower homologue in mediating competition, thus inhibiting tumor growth and spreading of pre-cancerous or cancerous cells within the tissue.

The ligands—particularly antibodies—described herein are useful for the diagnosis, prevention and therapy of cancer, particularly carcinoma. According to one embodiment of any ligand of the present invention, the ligand binds to a protein identified by one of SEQ ID NO 7, 8, 9, 10, 11, 12, 14, 15, 16 and/or 17 with an dissociation constant of below 50 nmol/l.

According to one embodiment of any of the aspects of the invention described herein which features a ligand to one of SEQ ID NO 7, 8, 9, 10, 11 or 12, the ligand is raised against the extracellular domains of a human protein isoform of Fwe, for example the extracellular C-terminal (SEQ ID NO 14) of SEQ ID NO 7, 8 or 10 or the predicted extracellular loops between transmembranes 1 and 2 (SEQ ID NO 15). Also the extracellular domain at the C-terminal (SEQ ID NO 16) of sequences ID 11 and 12 or the predicted extracellular loops between transmembranes 1 and 2 (SEQ ID NO 17) of sequences ID 11 and 12.

Antibodies or antibody fragments are particularly suitable embodiments of any ligand mentioned herein. Human (or humanized) immunoglobulin gamma antibodies are particularly useful. Humanized antibodies are antibodies derived from other species than homo sapiens, the protein sequences of which are modified to increase their similarity, and thus tolerability and physiologic function, in humans (Riechmann et al. (1988), Nature 332, 323-327 and publications citing this article).

According to a fifth aspect of the invention, an inhibiting nucleic acid molecule having a sequence complementary to one of SEQ ID NO 1, 2, 3, 4, 5, 6 or 13 and inhibiting the expression as a protein of a nucleic acid sequence identified by one of SEQ ID NO 1, 2, 3, 4, 5, 6 or 13 is provided for prevention or therapy of disease. Such inhibiting nucleic acid silences or “knocks down” the genetic message encoded by the sequences SEQ ID NO 1 to 6.

The art of silencing or “knocking down” genes, by degradation of mRNA or other effects, is well known. Examples of technologies developed for this purpose include siRNA, miRNA, shRNA, shmiRNA, and dsRNA. A comprehensive overview of this field can be found in Perrimon et al, Cold Spring Harbour Perspectives in Biology, 2010, 2, a003640.

Identity in the context of the present invention is a single quantitative parameter representing the result of a sequence comparison position by position. Methods of sequence comparison are known in the art; the BLAST algorithm available publicly is an example.

“Capable of forming a hybrid” in the context of the present invention relates to sequences that under the conditions existing within the cytosol of a mammalian cell, are able to bind selectively to their target sequence. Such hybridizing sequences may be contiguously reverse-complimentary to the target sequence, or may comprise gaps, mismatches or additional non-matching nucleotides. The minimal length for a sequence to be capable of forming a hybrid depends on its composition, with C or G nucleotides contributing more to the energy of binding than A or T/U nucleotides, and the backbone chemistry.

An inhibiting nucleic acid according to this fifth aspect may also be encoded an expression vector comprising a sequence encoding an interfering ribonucleic acid oligomer as described in the preceding paragraphs. Optionally, the sequence may be under the control of a promoter operable in mammalian cells. Such expression vectors facilitate production of an interfering RNA within the cell. Methods for making and using such expression vectors are known in the art.

Alternatively, an inhibiting nucleic acid molecule according to the above aspect of the invention may be a single-stranded or double-stranded antisense ribonucleic or deoxyribonucleic acid, comprising sequences complementary to an operon expressing one of SEQ ID NO 1, 2, 3, 4, 5, or 6 described above. Such operon sequences may include, without being restricted to, intron, exon, operator, ribosome binding site or enhancer sequences. Such antisense molecules may be 12-50 nucleotides in length.

“Nucleotides” in the context of the present invention are nucleic acid or nucleic acid analogue building blocks, oligomers of which are capable of forming selective hybrids with RNA oligomers (specifically with a sequence tract comprised in one or all of SEQ ID NO 1, 2, 3, 4, 5, or 6, such as a sequence tract comprised in SEQ ID NO 13) on the basis of base pairing. The term nucleotides in this context includes the classic ribonucleotide building blocks adenosine, guanosine, uridine (and ribosylthymin), cytidine, the classic deoxyribonucleotides deoxyadenosine, deoxyguanosine, thymidine, deoxyuridine and deoxycytidine. It further includes analogues of nucleic acids such as phosphotioates, 2′O-methylphosphothioates, peptide nucleic acids (PNA; N-(2-aminoethyl)-glycine units linked by peptide linkage, with the nucleobase attached to the alpha-carbon of the glycine) or locked nucleic acids (LNA; 2′O, 4′C methylene bridged RNA building blocks). The hybridizing sequence may be composed of any of the above nucleotides, or mixtures thereof.

The inventive inhibiting nucleic acid is able to abrogate the expression of one, two, three, four, five or all of the proteins identified by one of SEQ ID NO 7, 8, 9, 10, 11 and 12.

Such inhibiting nucleic acid molecule according to the fifth aspect of the invention may be a nucleic acid directed against and hybridizing to one, several or all of SEQ ID NO 1, 2, 3, 4, 5 or 6, preferentially with neutralizing properties. It may be a single-stranded or double-stranded ribonucleic acid oligomer or a precursor thereof, or a deoxyribonucleic acid or analogue thereof, comprising a sequence tract complementary to one, several or all of SEQ ID NO 1, 2, 3, 4, 5, or 6.

In one embodiment, the inhibiting nucleic acid molecule hybridizes to SEQ ID NO. 13.

In some embodiments, the hybridizing sequence of the inhibiting nucleic acid of the invention comprises 8, 9, 10, 11, 12, 13, 14, 15, 16, 17, 18, 19, 20, 21, 22, 23, 24, 25, 26, 27, 28, 29 or 30 nucleotides.

In some embodiments, the hybridizing sequence is at least 80%, 85%, 90%, 92%, 94%, 95%, 96%, 97%, 98% or 99% identical to the reverse complimentary sequence of one, several or all of SEQ ID NO 1, 2, 3, 4, 5, 6 or 13. In some embodiments, the hybridizing sequence comprises deoxyribonucleotides, phosphothioate deoxyribonucleotides, LNA and/or PNA nucleotides or mixtures thereof.

In some embodiments, the hybridizing sequence comprises ribonucleotides, phosphothioate and/or 2′-O-methyl-modified phosphothioate ribonucleotides.

In some embodiments, the hybridizing sequence comprises deoxyribonucleotides, phosphothioate deoxyribonucleotides, phosphothioate ribonucleotides and/or 2′-O-methyl-modified phosphothioate ribonucleotides.

In some embodiments, the inhibitory nucleic acid molecule comprises a cholesterol moiety or a peptide. In some embodiments, the hybridizing sequence is covalently attached to a cholesterol moiety or a peptide. Alternatively, the peptide may be part of a nucleic acid-peptide complex held together without covalent attachment by electrostatic or hydrophobic interaction. In some embodiments the inhibitory nucleic acid molecule comprises a peptide having or consisting of a TAT translocation sequence or a functional equivalent thereof.

In some embodiments, the above ribo- or deoxyribonucleotide moieties, optionally protected against degradation by phosphothioate linkage and/or 2′O-methyl ethers, and/or LNA or PNA moieties, are combined with cholesterol or peptide targeting and packaging moieties.

According to yet another aspect of the invention, a pharmaceutical composition is provided, wherein said pharmaceutical composition comprises a ligand, an inhibiting nucleic acid molecule and/or an expressed nucleic acid molecule according to any of the aspects of the invention outlined above.

In one embodiment, the pharmaceutical composition comprises a ligand, an inhibiting nucleic acid molecule and/or an expressed nucleic acid molecule specific for one isoform of human Fwe only.

In one embodiment, the pharmaceutical composition comprises one or several ligand(s), one or several inhibiting nucleic acid molecule(s) and/or one or several expressed nucleic acid molecule(s) so that the pharmaceutical composition in total inhibits several or all isoforms of human Fwe. The data of the present invention show that abrogation of all Fwe signalling in the mouse carcinoma model confers great advantages. Thus, according to this embodiment, the therapeutic approach is to downregulate all Fwe isoforms, (as in the KO mouse of the examples), targeting, for example, their conserved sequences on protein or nucleic acid level, thus reducing or suppressing the expression of all isoforms in the tissues surrounding the tumor.

In one embodiment, the pharmaceutical composition comprises one or several ligand(s), one or several inhibiting nucleic acid molecule(s) and/or one or several expressed nucleic acid molecule(s) so that the pharmaceutical composition in total inhibits the isoforms of human Few encoded or represented by SEQ ID NO. 2, SEQ ID NO. 3 and/or SEQ ID NO. 6, and/or SEQ ID NO. 7, SEQ ID NO. 8 and/or SEQ ID NO. 12, respectively. These are the closest relatives, measured by nucleic acid or protein identity or similarity, to murine isoforms 1 and 3, which the examples of the present invention illustrate are a “loser” form of Fwe (see FIG. 9 and Examples).

The pharmaceutical compositions of the invention are particularly useful for the prevention or treatment of cancer.

Similarly within the scope of the present invention is a method of preventing or treating cancer in a patient in need thereof, comprising administering to the patient a ligand, inhibiting nucleic acid molecule or pharmaceutical composition according to the invention.

Similarly, a dosage form for the prevention or treatment of cancer is provided, comprising a ligand, inhibiting nucleic acid molecule or pharmaceutical composition according to one of the above aspects of the invention. Dosage forms may be for enteral administration, such as nasal, buccal, rectal, transdermal or oral administration, or as an inhalation form or suppository. Alternatively, parenteral administration may be used, such as subcutaneous, intravenous, intrahepatic or intramuscular injection forms. Optionally, a pharmaceutically acceptable carrier and/or excipient may be present.

Furthermore, a method is provided for identifying a candidate compound for development of a drug suitable for the prevention and/or treatment of cancer disease is provided, said method comprising:
a) incubating a mammalian cell in the presence of a compound that is to be examined, subsequently
b) determining as an expression level within said mammalian cell the quantity of
   i) a nucleic acid sequence identified by SEQ ID NO 1, 2, 3, 4, 5, 6 and/or 13, or
   ii) an amino acid sequence SEQ ID NO 7, 8, 9, 10, 11, 12, 14, 15, 16 and/or 17
c) comparing said expression level to a standard, and
d) assigning to said compound a likelihood to serve as a candidate for cancer drug development as a function of deviation of said expression level from said standard.

According to another aspect of the invention, a method for identifying a candidate compound for development of a drug suitable for the prevention and/or treatment of cancer disease is provided, said method comprising:
a) incubating a mammalian cell in the presence of a compound that is to be examined,
b) determining as an expression level within said mammalian cell the quantity of
   i) a nucleic acid sequence identified by SEQ ID NO 1, 2, 3, 4, 5, 6 and/or 13, or
   ii) an amino acid sequence SEQ ID NO 7, 8, 9, 10, 11, 12, 14, 15, 16 and/or 17
c) determining whether said expression level is above or below a predetermined cut-off level or comparing said expression level to a standard value,
d) identifying said compound suitable for the prevention and/or treatment of cancer disease evaluating the result of step c), or assigning to said compound a likelihood to serve as a candidate for cancer drug development.

A compound that inhibits the expression of the mammalian homologues of Flower is a drug candidate for the prevention and/or treatment of cancer.

Wherever alternatives for single features such as, for example, an isotype protein or coding sequence, ligand type or medical indication are laid out herein as “embodiments”, it is to be understood that such alternatives may be combined freely to form discrete embodiments of the invention disclosed herein. Thus, any of the alternative embodiments for a detectable label may be combined with any of the alternative embodiments of ligand and these combinations may be combined with any medical indication or diagnostic method mentioned herein.

The invention is further illustrated by the following examples and figures, from which further embodiments and advantages can be drawn.

### DESCRIPTION OF THE FIGURES

FIG. 1 shows the mFwe gene locus, protein isoforms and their over-expression in Drosophila wing imaginal discs. (A) Schematic representation of mFwe gene locus showing chromosome location and protein-coding alternative splice transcripts. Exon coding sequence is indicated with black boxes, untranslated sequences with white boxes. Exons are assigned a number, alternative exons are assigned a number and a letter. The Ensembl transcript ID number is provided next to each transcript. A box outline marked “Ex. 3” indicates the common exon that is targeted for deletion. (B) A cartoon displaying membrane topology prediction for the four mFwe protein isoforms using SOSUI algorithm. A number indicates identical transmembrane domains. Protein domains that are encoded by exon 3 are grey. (C) Expression of HA-tagged mFwe proteins in Drosophila wing imaginal discs. Confocal fluorescence microscopy images of Drosophila wing imaginal discs stained with α-HA antibody. Expression of mFwe proteins is induced by hh-GAL4, whose expression is restricted to the posterior EGFP-marked compartment. The images show over-expression of dFwe(LoseA)-HA and mFwe 4-HA. Panels to the right show Z-sections of the same wing imaginal discs to visualize distribution of the proteins along the apico-basal axis of the epithelium. Magnification: 20×, 40×.

FIG. 2 shows an analysis of mFwe isoforms by gain-of-function assays in Drosophila and quantification of mFwe transcripts in skin papillomas and papilloma-surrounding skin. (A) Assay of mFwe function by generation of random gain-of-function clones of cells in Drosophila wing imaginal discs. Confocal fluorescence microscopy images of Drosophila wing imaginal discs with Act>gal4 EGFP-marked clones that over-express the indicated transgenes at 72 hours after clone induction (ACI). DAPI (blue), EGFP (green). Magnification: 20×. (B) Average area occupied by Act>gal4 EGFP-marked clones, represented as percent of total disc area, at 72 hours after clone induction (ACI). The percent area occupied by the clones of each genotype is compared to the LacZ-expressing clones (negative control): statistical significant difference (* p<0.05, Student's t test) is found for comparisons of UASmFwe9 1-HA and UASIacZ, UASmFwe3-HA and UASIacZ, UASdFwe(LoseA)-HA and UASIacZ. Data are means±s.e.m. For each transgene, at least 20 wing imaginal discs were analyzed. (C) Confocal microscopy image of activated caspase 3 (C3) immunofluorescence staining in Drosophila wing imaginal discs where mFwe1-HA is over-expressed in EGFP-marked clones of cells. The image to the left shows fluorescence signal from activated caspase 3 only, with delineation of EGFP-marked clones by white contours. (D) Confocal microscopy image of activated caspase 3 (C3) immunofluorescence staining in Drosophila wing imaginal disc posterior compartment marked by EGFP. The image to the left shows fluorescence signal from activated caspase 3 only, with delineation of the posterior compartment by white contours. (E) Real-time quantitative PCR analyses of the expression of mFwe 1 transcript in wild-type papilloma and corresponding papilloma-surrounding skin samples. The plot represents fold change in mFwe 1 expression relative to its expression in wild-type skin of age-matched mice not treated with DMBA/TPA. The mRNA expression was previously normalized to the expression of the b-actin housekeeping gene. The data represent means±s.e.m of analyses of samples from three mice per condition. * p<0.05, Student t test of comparison between expression level in papilloma-surrounding skin (black) and untreated skin (grey).

FIG. 3 shows the generation of mFwe constitutive knock-out mice. (A) Schematic outline of the gene targeting strategy used to generate mFwe constitutive knock-out mice. Solid black line represents chromosome sequence; black and white rectangles represent coding and noncoding exons, respectively. Alternative exons are represented above the locus with grey/white rectangles. The translation initiation codon (ATG) and the stop codon (STOP) are indicated. loxP sequences are represented by red triangles. PGK-Neo positive selection cassette is indicated with a blue box. FRT sequences are represented by double magenta triangles. NheI restriction sites for 3′ Southern blot strategy are indicated by grey vertical lines. Grey horizontal lines indicate the length and location of the DNA fragments produced upon digestion with NheI enzyme during the Southern blot procedure. The length of these fragments is indicative of a mFwe WT and KO alleles. The location of the external 3′ probe for Southern blot is indicated with a red box. The forward and reverse primers used for PCR genotyping are indicated with white triangles. Black horizontal lines indicate length and position of PCR fragments corresponding to the WT and mutant alleles. To generate constitutive knock-out mice, the loxP-flanked exon 3 (floxed allele) is removed by crossing to a germline::Cre deleter mouse. (B) Southern blot verification of the presence of the mFwe mutant (Dex3) allele. (C) PCR genotyping of mice carrying the mFwe Dex3 allele. (D) Semi-quantitative RT-PCR analyses of mFwe transcript expression in the indicated tissues using primers complementary to exon 3 and exon 4 confirms the absence of mFwe mRNA expression in the mFwe mutant animals and its decreased expression in mFwe(+/Dex3) mice. Amplification of Gapdh cDNA serves as an internal PCR control.

FIG. 4 shows that mFwe knock-out mice are resistant to DMBA/TPA-induced skin carcinogenesis. Eight- to twelve-week-old mFwe(+/+), mFwe(Δex3/+) and mFwe(Δex3/Δex3) mice were subjected to two-step chemical carcinogenesis with DMBA and TPA. (A) Timing of DMBA/TPA skin carcinogenesis protocol. (B) Dorsal view of mFwe(+/+ and mFwe(Δex3/Δex3) mice after 15 weeks of DMBA/TPA treatment. (C) Average number of papillomas per mouse. The difference in average tumor number between wild-type and KO or between heterozygous and KO mice becomes significant from week 10 onwards; * p<0.05, Student's t test. From week 13 onwards the difference between the number of papillomas in wild-type and in mFwe (Δex3/Δex3) mice is also significant according to Mann-Whitney test: p<0.05. (D) Tumor incidence. Comparison of mFwe(+/+) and mFwe(Δex3/Δex3) incidence curves: p=0.06 (log-rank test) and p<0.05 (Gehan-Breslow-Wilcoxon test). (E) Immunohistochemical analyses of mFwe (Δex3/Δex3) papillomas and papilloma-surrounding epidermis. Quantification of Ki67-positive area in papilloma and papilloma-surrounding epidermis of mFwe(+/+) and mFwe(Δex3/Δex3) mice is shown to the right. Papilloma-surrounding skin is normal-looking skin that occupies 1000 μm at each side of the corresponding tumor analyzed. Data represent measurements from five mice per genotype. Bars are s.e.m. Panels to the left show representative images of the immunostainings. Brown color indicates immunostaining signal. Insets show magnified images of regions from the corresponding tissue. Red arrowheads indicate Ki67-positive cells at the basal layer of the skin epidermis. Scalebars, 200 and 100 μm.

FIG. 5 shows the expression of mFwe transcripts in tissues of adult wild-type mice and in skin papillomas and papilloma-surrounding skin of mice treated with DMBA/TPA. (A) Plots showing the expression level of mFwe alternative transcripts mFwe 1 (A) mFwe 2 (B), mFwe 3 (C), mFwe 4 (D) in the indicated tissues of C57BL/6

| | mFwe 1 | mFwe 2 | mFwe 3 | mFwe 4 |
|---|---|---|---|---|
| Untreated | 1.45 × 10−6 | 1.60 × 10−6 | 9.00 × 10−8 | 1.62 × 10−8 |
| skin | | | | |
| Papilloma | 4.06 × 10−6 | 3.59 × 10−6 | 1.67 × 10−7 | 3.07 × 10−8 |
| Papilloma- | 7.96 × 10−6 | 5.76 × 10−6 | 2.70 × 10−7 | 4.04 × 10−8 |
| surrounding | | | | |
| skin | | | | |

adult mice measured by real-time quantitative PCR. The data represent average mRNA expression level relative to the expression of the 18S rRNA gene of three independent experiments. Bars are means±s.e.m. Y-axes are linear in scale, the scale for A-D is 10E-4 for the upper most bar and 0 for the lower end. The dotted lines mark the level of expression of mFwe 1 and mFwe 2 in papilloma-surrounding skin of mice treated with DMBA/TPA, which is presented the following table:

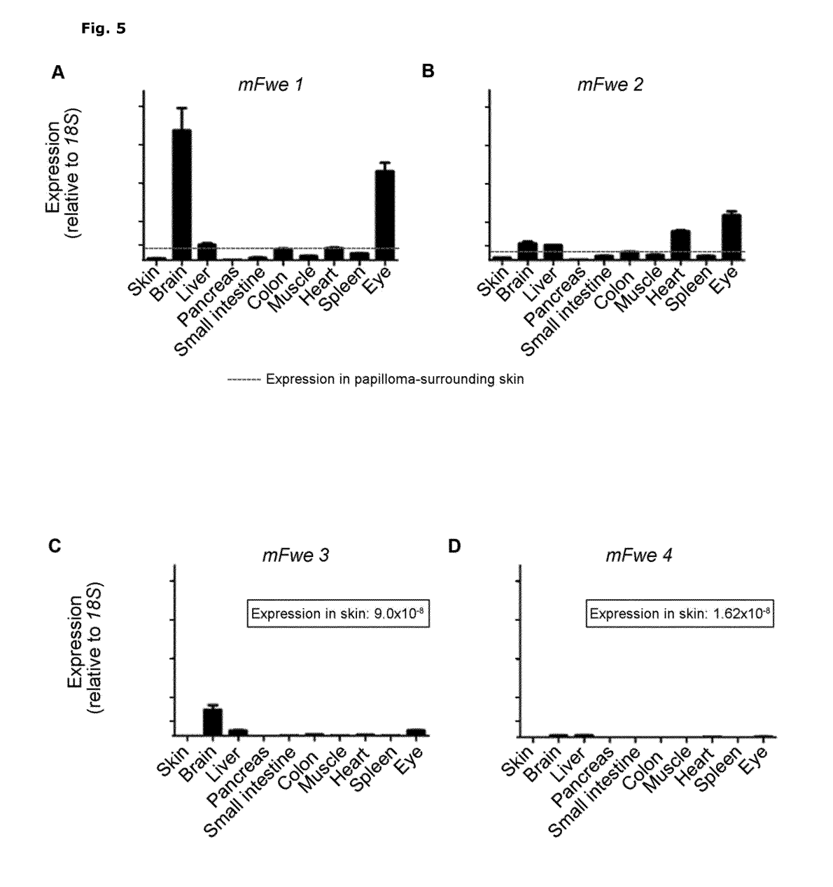

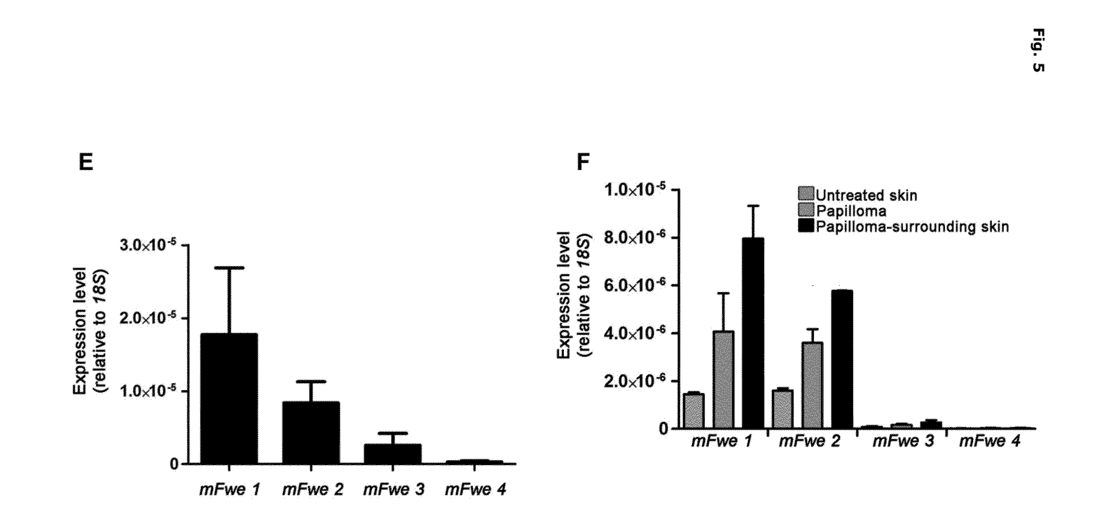

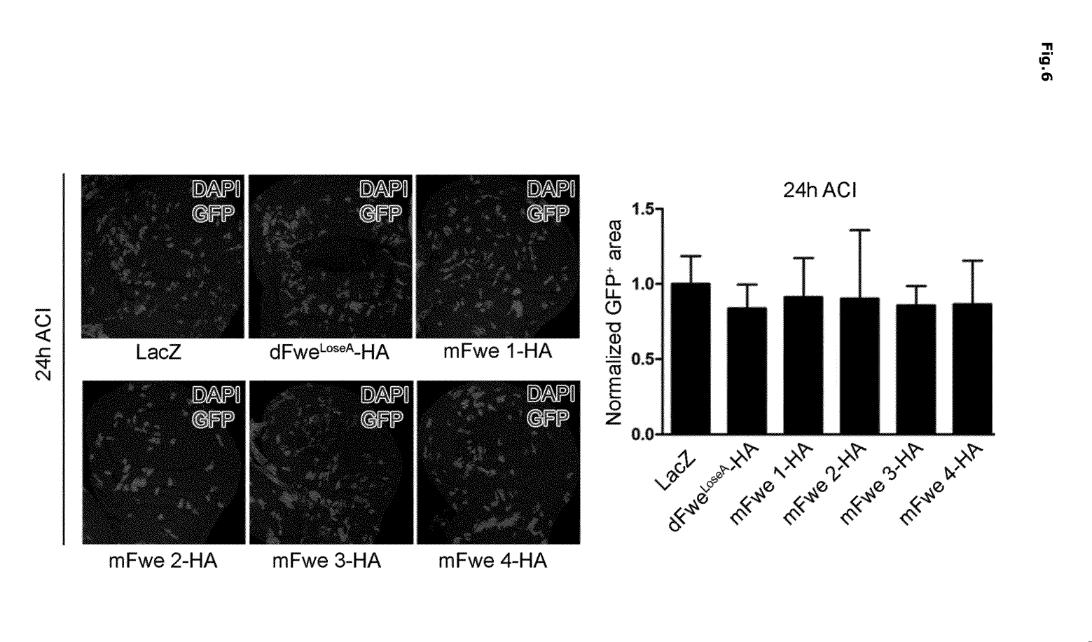

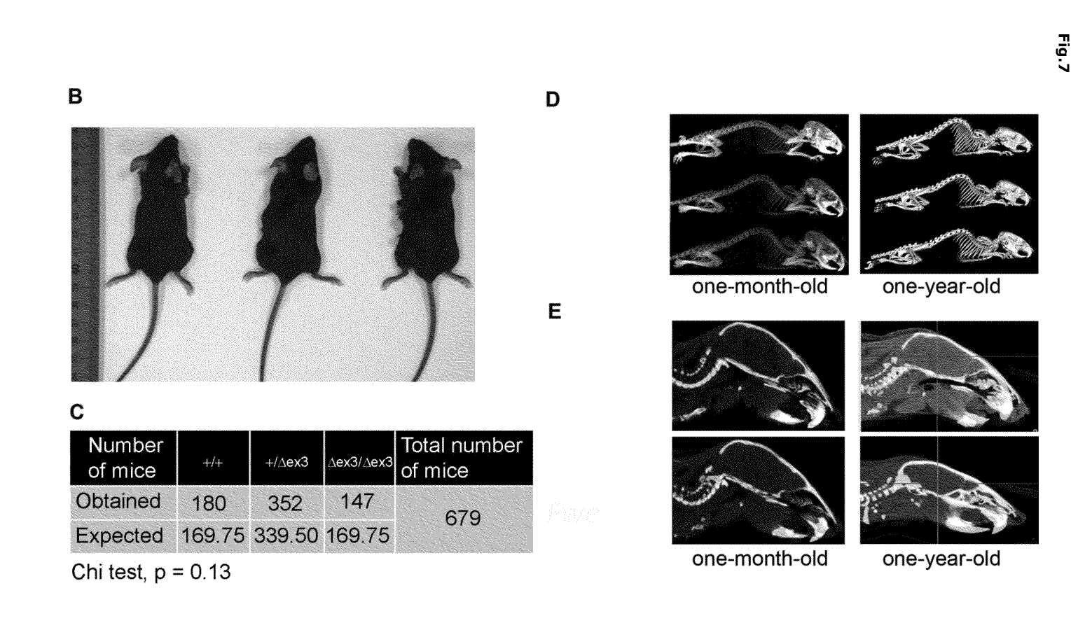

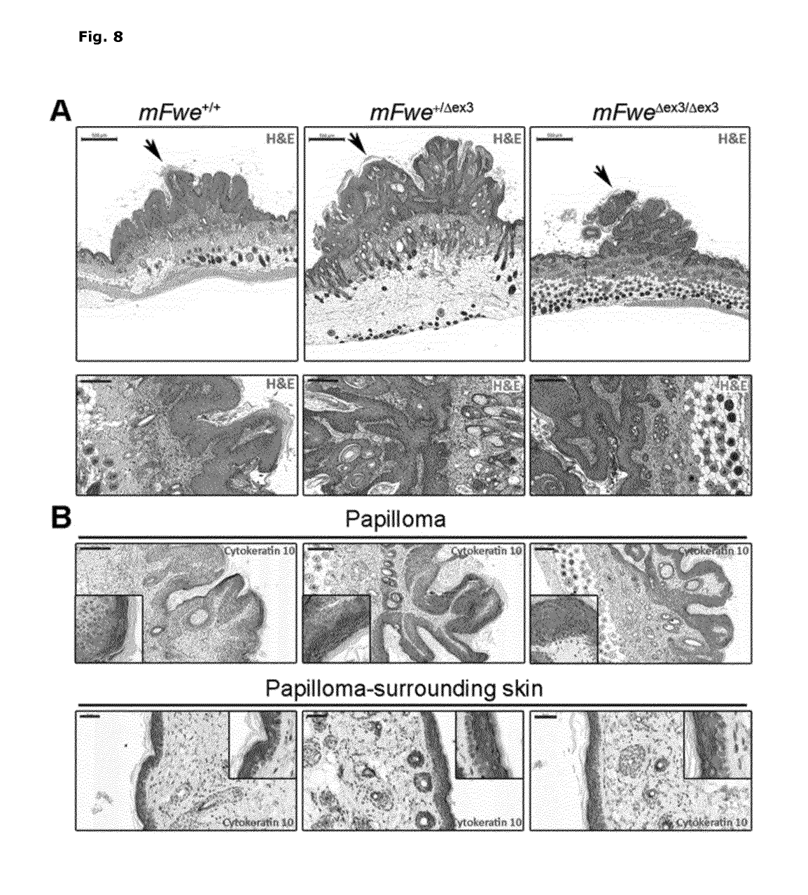

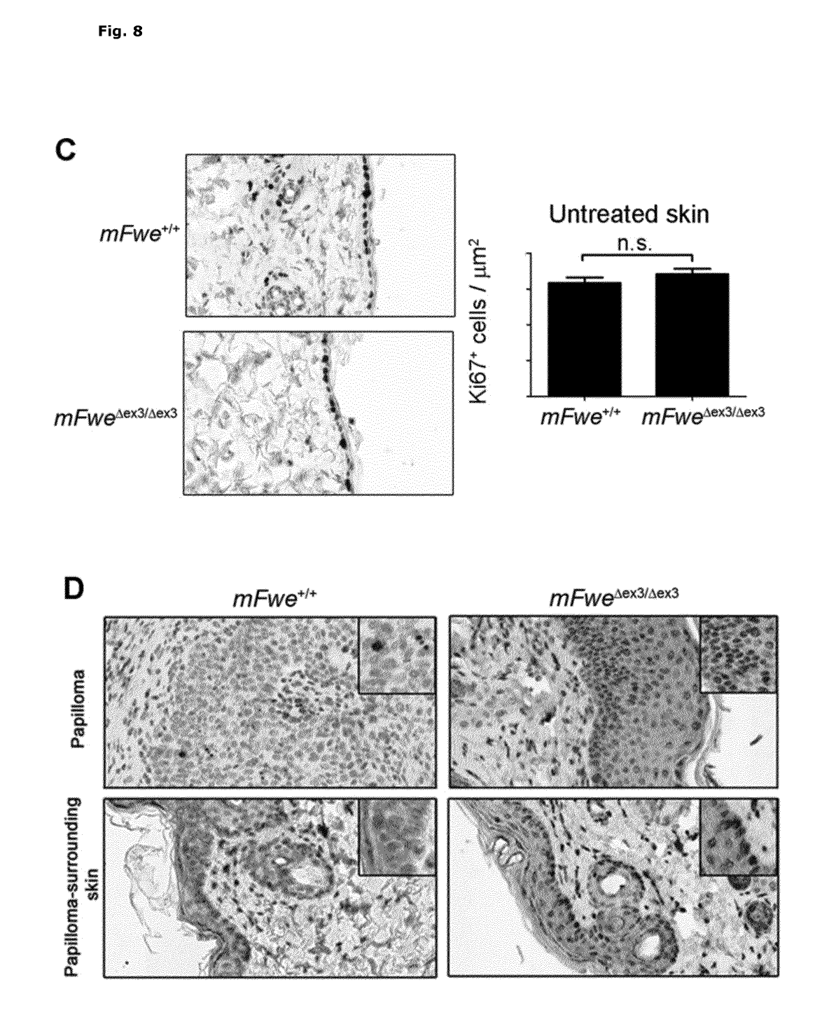

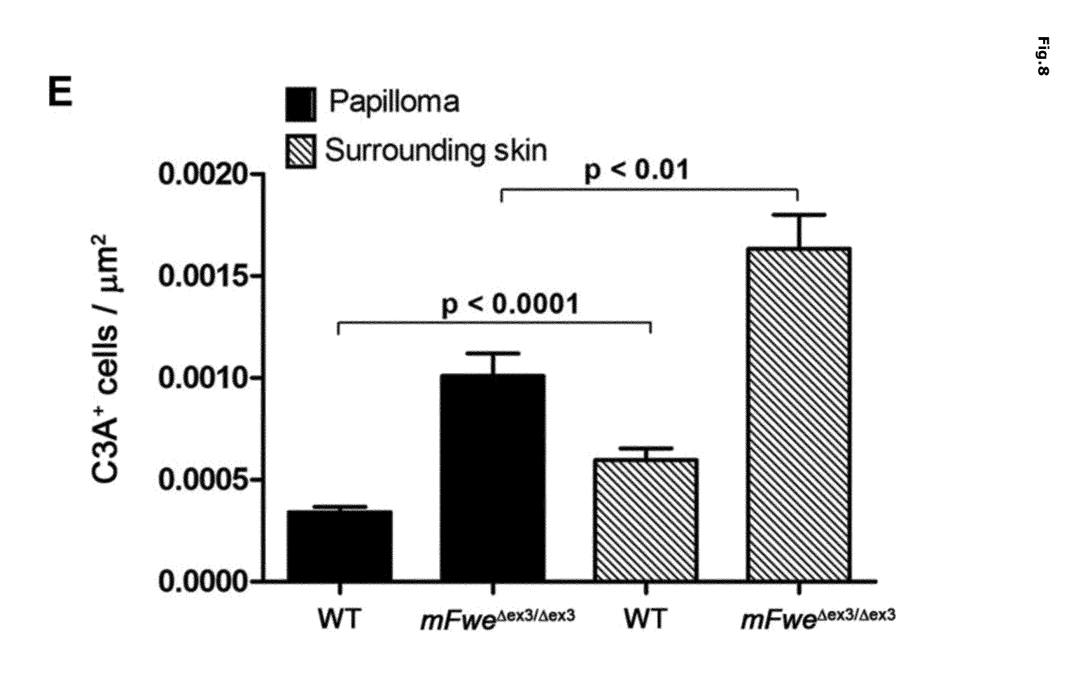

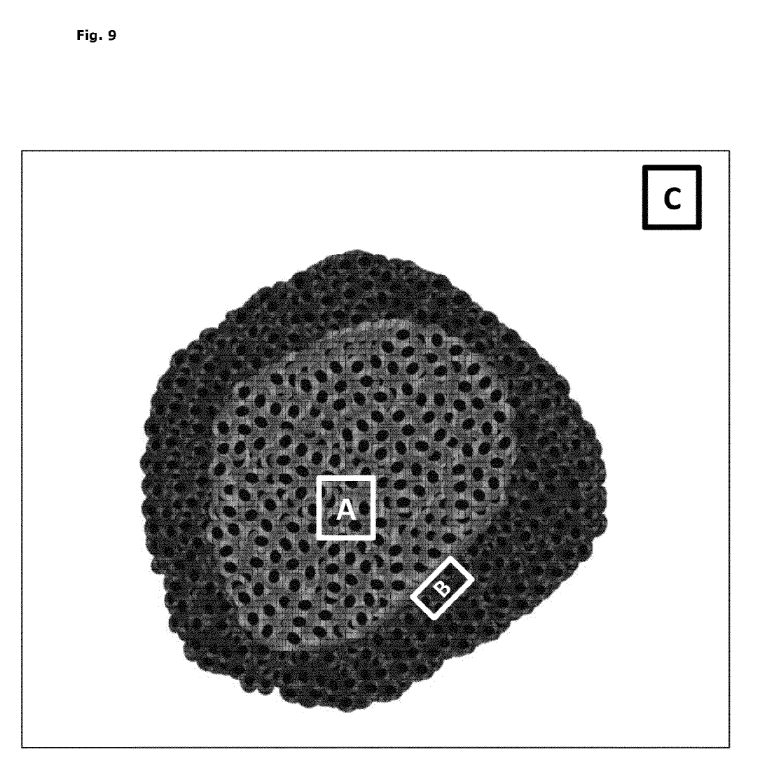

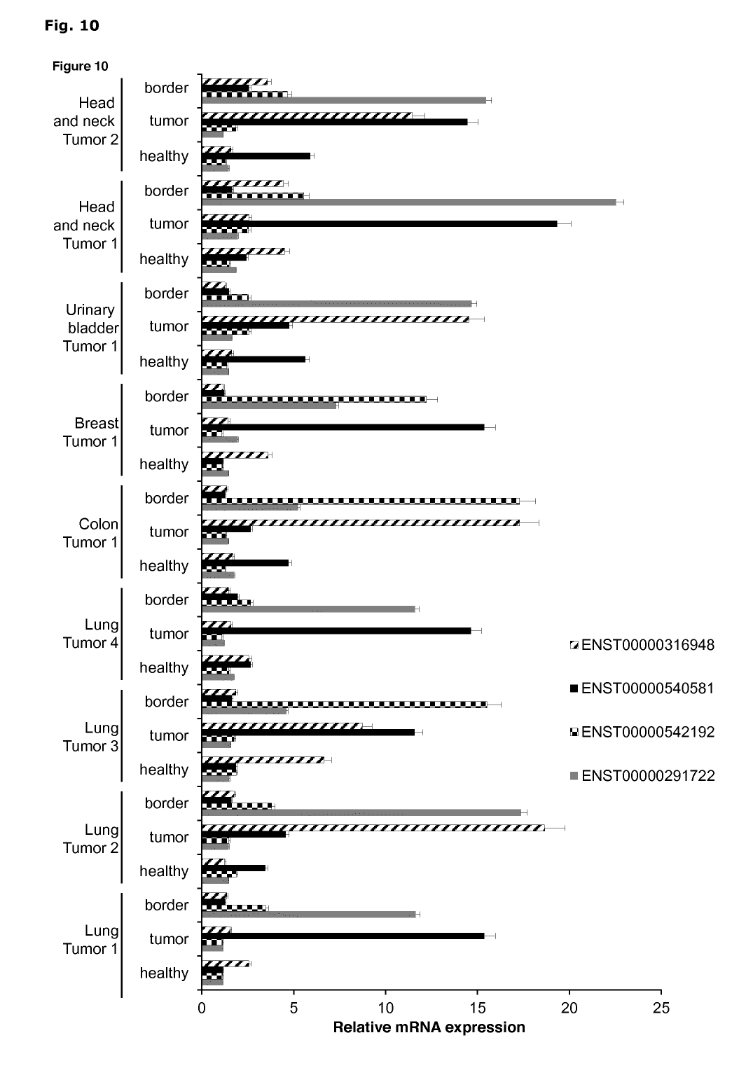

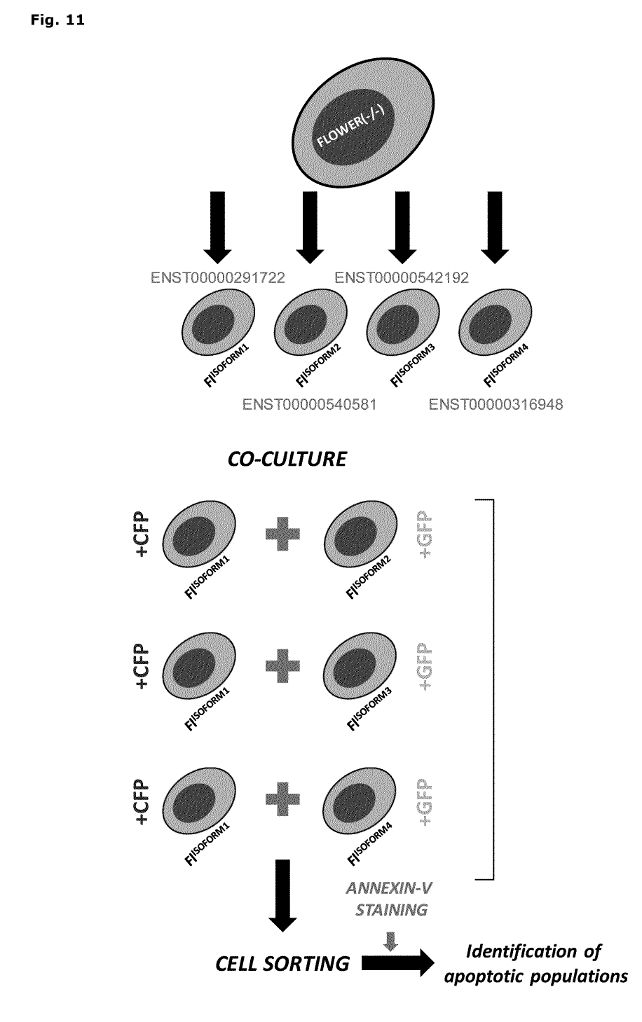

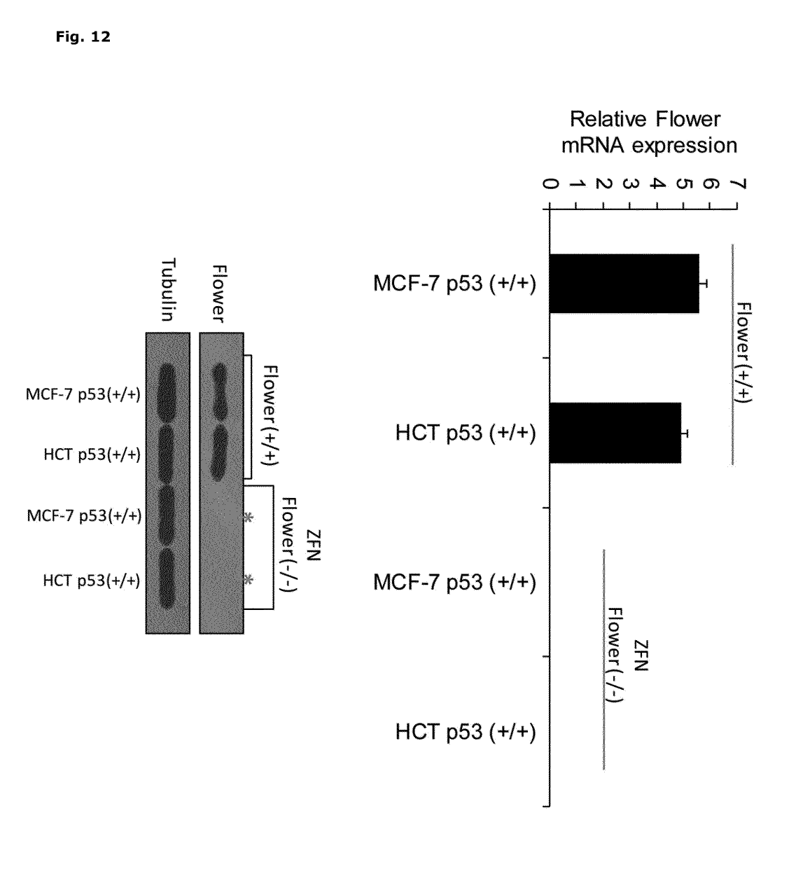

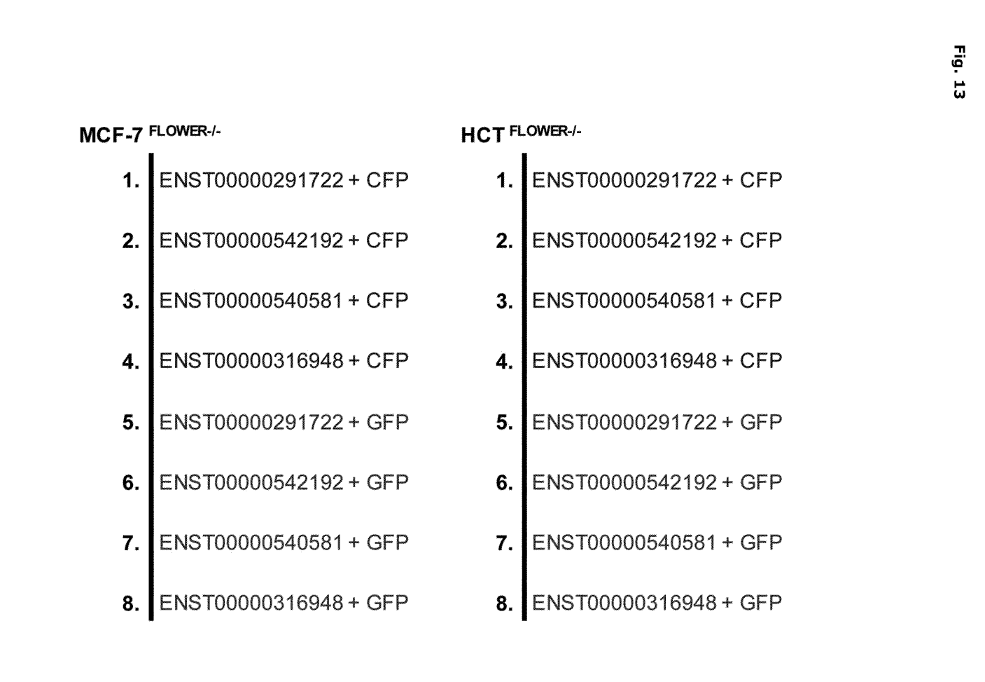

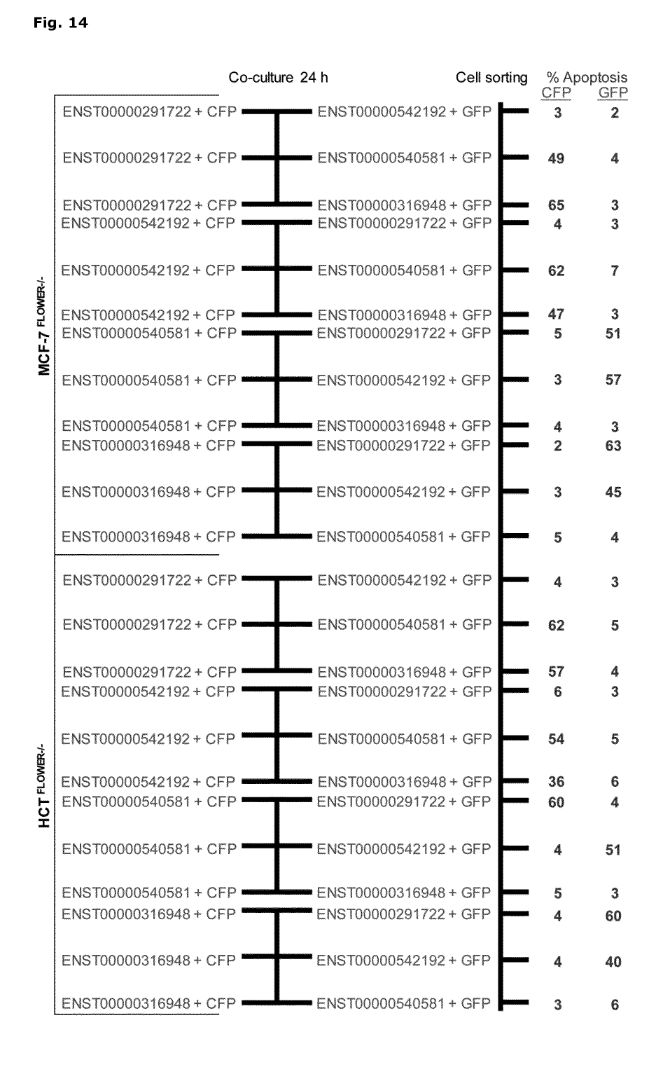

FIG. 5 (E) Average expression level of mFwe transcripts in the ten tissues analyzed in A-D. Bars are means±s.e.m. (F) Real-time quantitative PCR analyses of the expression of mFwe 1, mFwe 2, mFwe 3, and mFwe 4 transcripts in wild-type papilloma, papilloma-surrounding skin and skin of mice not treated with DMBA/TPA. Tissue samples were obtained from mice of the same age. The data is normalized to the expression of the 18S housekeeping gene. The values of expression for each sample are summarized in the table below the graph. The data represent means±s.e.m of analyses of samples from three mice per condition. p values were determined using Student's t test: for mFwe1 p<0.05 (untreated—papilloma surrounding skin); for mFwe2 p<0.0001 (untreated—papilloma surrounding skin), p<0.05 (papilloma v. papilloma surrounding skin).

FIG. 6 shows the analysis of mFwe isoforms by gain-of-function assays in Drosophila (A) Assay of mFwe function by generation of random gain-of-function clones of cells in Drosophila wing imaginal discs. Confocal fluorescence microscopy images of Drosophila wing imaginal discs with Act>gal4 EGFP-marked clones that over-express the indicated transgenes at 24 hours after clone induction (ACI). DAPI (blue), EGFP (green). Magnification: 20×. The graphic to the right represents average area occupied by Act>yellow>gal4 EGFP-marked clones, represented as percent of total disc area, at 24 hours after clone induction (ACI). The percent area occupied by the clones of each genotype is compared to the LacZ-expressing clones (negative control). For each transgene, at least 20 wing imaginal discs were analyzed. No statistical significant difference was found.

FIG. 7 shows that mFwe-deficient mice display a normal phenotype. (A) mFwe cDNA obtained from mFwe(Δex3/Δex3) mice was sequenced. The obtained DNA sequence is presented together with the corresponding protein sequence. Deletion of exon 3 results in a frameshift, which generates a new mRNA splice site (black triangle) between exon 1 and exon 4 and a premature stop codon (box) in exon 4. Translation of this sequence gives rise to a truncated protein (41 amino acids). Amino acids are represented with a single-letter code. Numbers to the left and right of each line indicate sequence length and nucleotide/amino acid position. The sequences are given in SEQ ID NO 43 and 44. (B) mFwe(Δex3/Δex3) mice have a normal stature and external phenotype (left to right: mFwe(+/+); mFweΔex3/+; mFweΔex3/Δex3) (C) Embryonic lethality of mFweΔex3/Δex3 mice was not observed since the number of mFwe+/+, mFweΔex3/+ and mFweΔex3/Δex3 mice born from a total of 93 matings between mFweΔex3/+ mice is in a proportion similar to the expected Mendelian one. (D) Analysis of skeletal morphology of mFweΔex3/Δex3 mice over time did not reveal any difference. Micro-computed tomography images showing lateral view of the skeleton of female littermates of the indicated genotypes at one month of age and at one year of age. (top to bottom: mFweΔex3/Δex3; mFweΔex3/+; mFwe(+/+)) (E) Superposition of micro-computed tomography images of the skull of female littermates at one month of age and at one year of age. Top: wild-type (grey) versus knock-out (white) and bottom: wild-type (grey) versus heterozygous (white).

FIG. 8 shows histological and immunohistochemical analyses of skin papillomas induced by DMBA/TPA treatment. (A) Representative hematoxylin- and eosin-stained sections from papillomas of mFwe(+/+), mFwe(Δex3/+) and mFwe(Δex3/Δex3) mice. Papillomas are indicated by black arrows. Scalebars, 500 and 200 μm. (B) Representative sections of papillomas and normal skin epidermis adjacent to the corresponding papilloma of mFwe(+/+), mFwe(Δex3/+) and mFwe(Δex3/Δex3) mice stained with an antibody against the keratinocyte differentiation marker cytokeratin 10. Insets show magnified images of regions of the same papillomas or epidermis. Brown color indicates immunostaining signal. Scalebars, 200 and 50 μm. (C) Quantification of Ki67-positive cells in skin epidermis of mFwe(+/+) (left column) and mFwe(Δex3/Δex3) (right column) mice not treated with DMBA/TPA. The difference in the number of Ki67-positive cells between mFwe(+/+) and mFwe(Δex3/Δex3) skin is not significant (n.s., Student's t test). Bars are s.e.m. Panels to the left show representative images of the immunostainings (top panel mFwe(+/+));) (bottom panel mFwe(Δex3/Δex3). Brown color indicates immunostaining signal. Magnification 40×. (D) Immunohistochemical staining of activated caspase 3 (C3A) in papillomas and in papilloma-surrounding skin of mFwe(+/+) and mFwe(Δex3/Δex3) mice. Papilloma-surrounding skin is normal-looking skin that occupies 1000 μm at each side of each tumor analyzed. Brown color indicates immunostaining signal. Insets show magnified images of regions from the corresponding tissue. Magnification is 40×. (E) Quantification of activated caspase 3-positive cells in papilloma and in papilloma-surrounding skin of mFwe(+/+) and mFwe(Δex3/Δex3) mice. From left to right: black columns: Papilloma; grey: surrounding skin; left WT right mFwe(Δex3/Δex3). Data represent measurements from 3 mice per genotype. Bars are s.e.m. p values are of the indicated comparisons using Student's t test. The table below summarizes the data and indicates the ratio between the average number of C3A-positive cells in the papilloma-surrounding skin and the papillomas:

Average number of C3A+ cells/μm2

| | WT | mFweΔex3/Δex3 |
|---|---|---|
| Papilloma | 0.0003415 | 0.001009 |
| Surrounding skin | 0.0005959 | 0.001633 |
| Ratio* | 1.74 | 1.62 |

*Ratio between the average number of C3A+ cells in papilloma-surrounding skin and in papillomas.

FIG. 9 shows a schematic representation of the zones from and around the tumor from where the tissue samples were collected (see Example 7). (A) is tumor tissue; (B) is tumor boundary host tissue; (C) is healthy tissue.

FIG. 10 shows expression profiles for Fwe isoforms in different tissue samples. Bars give the four isoform expression levels next to any location indication in the order—from top to bottom—ENST00000316948 (diagonal stripes), ENST00000540581 (solid black), ENST00000542192 (chequered), ENST00000291722 solid grey).

FIG. 11 shows the research strategy of Example 8. Flower-negative cultured cells transfected with constructs expressing the indicated isoforms (SEQ ID No 2, 6, 1, 5) and either cyan fluorescent protein (CFP) or green fluorescent protein (GFP). All dual isoform combinations of CFP and GFP expressing cells are co-cultured and subsequently stained with annexin-V for induction of apoptosis. The (+/+) staining indicates the loser population; the respective Flower Isoform is identified as the Flower(LOSE) isoform; the other population behaves as the winner and the respective Flower isoform is identified as the Flower(UBI) isoform.

FIG. 12 shows a western blot experiment demonstrating the successful knockout of the Flower gene from human MCF-7 and Human HCT116 cells. Lane 1 represents the expression of Flower protein in MCF-7 p53 (+/+) cells, lane 2 represents the expression of flower protein in HCT116 p53 (+/+) cells. In lane 3 and lane 4 the MCF-7 and HCT cells are processed with ZFN CKOZFN5222 to knockout the Flower gene from the genome. The western blot analysis of Flower protein using an anti-flower polyclonal antibody shows that the expression of Flower protein is abolished from the cells. This data suggests efficient knockout of flower in these cell lines.

FIG. 13 shows the co-culturing scheme used in Example 8.

FIG. 14 shows the apoptotic fractions of the cell lines co-cultured in Example 8.

### EXAMPLES

Materials and methods used in Examples:

### Generation of Fwe Knock-Out Mice

The Fwe knock-out mouse was developed using genOway technical services (genOway, Lyon, France). The targeting vectors for generation of Fwe null alleles were constructed by using genomic DNA (21.3 kb) that encompasses the entire murine Fwe gene region isolated from a 129Sv/Pas miniBAC library. The targeting vector used for homologous recombination consisted of asymmetric homology arms isogenic with the ES cell line of 129Sv/Pas genetic background. The linearized targeting construct (40 μg) was introduced into 129Sv/Pas mouse embryonic stem cells (5×106 cells) by electroporation (260 V, 500 μF). Genomic DNA extracted from the amplified ES cell clones was screened for homologous recombination by both PCR and Southern blot strategies. The 3′ Southern blot screening is based on digestion of genomic DNA with NheI and hybridization of an external 523 bp probe downstream of the 3′ homology sequence of the Fwe targeting vector. ES cells from positive ES cell clones were microinjected in C57BL/6 blastocysts, which were then introduced into pseudo-pregnant OF1 female mice. Highly chimaeric males (80% chimaerism) were mated with C57BL/6 wild-type females to investigate whether the recombined ES cells contribute to the germ-line. The resulting F1 animals that showed agouti coat color were heterozygous for the recombined allele and were in 1:1 mixed genetic background C57BL/6:129 Sv/Pas. To generate constitutive knock-out mice the loxP-flanked exon 3 (floxed allele) was removed by crossing to a germline::Cre deleter mouse. Constitutive Fwe knockout mice were screened by PCR using the following primers: FW 5′-CTAACTACCCAAGCATCCTG-3′ (SEQ ID NO 18), RVex4 5′-CGCAGTTGAAGAGTCCAGAG-3′ (SEQ ID NO 19), and RVex3 5′-TACACCAAAGAATGACCCAC-3′ (SEQ ID NO 20), which yield 685 and 354 bp products for the mutant and wild-type alleles, respectively.

### Mouse Maintenance and Breeding

The mice used in this study were housed in specific pathogen-free animal facility at the Spanish National Cancer Research Center (Madrid). The animals were maintained by crossing to mice of C57BL/6J genetic background. The experiments were performed using littermate mice.

### Induction of Skin Papillomas

The back skin of two-to three-month old mFwe(+/+), mFwe(Δex3/+) and mFwe(Δex3/Δex3) littermate mice was shaved and one day later was painted with a single dose of 25 μg of 7,12-dimethylbenz[a]anthracene (Sigma) dissolved in 200 μl acetone. Two days later, tumor growth was promoted by applying 12.5 μg of 12-O-tetradecanoylphorbol-13-acetate (Calbiochem) dissolved in 200 μl acetone twice a week for a period of 15 weeks. The mice were observed every three days and size, number and characteristics of the skin lesions were annotated. Measurement of tumor size was done twice per week using a digital caliper.

### Histology and Immunohistochemistry

Skin papillomas and surrounding skin were fixed in neutral-buffered formalin during 24 hours and subsequently embedded in paraffin. Sections of ˜5 μm were cut and stained with hematoxylin and eosin following standard procedures. For immunohistochemistry, ˜5 μm sections from formalin-fixed, paraffin-embedded tissue samples were incubated with anti-Ki67 antibody (TEC-3, DAKO). The quantification of immunohistochemistry samples was performed automatically using AxioVision software (Carl Zeiss, Germany) by measuring the area occupied by Ki67-positive cells in papillomas and the papillomasurrounding epidermis (1000 μm at each side of a papilloma).

### RNA Isolation and Quantitative RT-PCR

Total RNA of mouse tissues was extracted using Trizol reagent (Invitrogen) following the manufacturer's instructions. It was treated with DNase I (Promega) and additionally purified using Qiagen RNeasy columns. cDNA was synthesized using SuperScript II reverse transcriptase (Invitrogen). Semi-quantitative PCR was done using mFwe-specific primer that hybridizes to mFwe exon 3 5′-CTCTTCAACTGCGTCACTAT-3′ (SEQ ID NO 21), a primer that hybridizes to mFwe exon 4 5′-TGCCCACTGCTATCAAATAA-3′ (SEQ ID NO 22) and Gapdh-specific primers 5′-GTATGTCGTGGAGTCTACTG-3′ (SEQ ID NO 23) and 5′-TCATCATACTTGGCAGGTTT-3′ (SEQ ID NO 24). To quantify the abundance of mFwe transcripts in wild-type mice treated with the DMBA/TPA carcinogenesis protocol, total RNA was extracted separately from skin papillomas and the corresponding papilloma-surrounding skin and was analyzed by real-time quantitative PCR. The expression level of each transcript in both samples was compared to its expression in the skin of age-matched wild-type mice not treated with DMBA/TPA. Papilloma-surrounding skin was a normal-looking skin located within a diameter of approximately 1 cm from a papilloma. To determine the expression level of mFwe transcripts in different tissues of wild-type mice, total RNA was extracted from skin, brain, liver, pancreas, small intestine, colon, muscle, heart, spleen and eye tissue samples and was analyzed by real-time quantitative PCR. Real-time quantitative PCR was performed using 0.5 μl of cDNA prepared from 3 μg of total RNA, 2× Power SYBR green PCR master mix (Applied Biosystems) and BioRad Single-Color PCR detection apparatus. All PCR reactions were set up in triplicates and the experiments were performed with at least three different samples. Data were analyzed using the comparative C_{T} method (Schmittgen and Livak, (2008), Nat Prot 3, 1101-1108). The Ct values of samples and controls were normalized to the expression level of 18S endogenous housekeeping gene. The primers used were 18S-Fw 5′-GTAACCCGTTGAACCCCATT-3′ (SEQ ID NO 25), 18S-Rv 5′-CCATCCAATCGGTAGTAGCG-3′ (SEQ ID NO 26), mFwe1-Fw 5′-TCCACACTTCTCTGGTTCTG-3′ (SEQ ID NO 27), mFwe1-Rv 5′-GTGAGTACTGCTGTCTAGCC-3′ (SEQ ID NO 28), mFwe2-Fw 5′-CGATGCCATTTCTTATGCTC-3′ (SEQ ID NO 29), mFwe2-Rv 5′-TGACACTCAGTCTTCTCCAG-3′ (SEQ ID NO 30), mFwe3-Fw 5′-CAAACACAGTAGCTGAGAAGG-3′ (SEQ ID NO 31), mFwe3-Rv 5′-TAGAGGGAAATGGTGTTTCTG-3′ (SEQ ID NO 32), and mFwe4-Fw 5′-GTTTGCTAAATCCTGGGTGTC-3′ (SEQ ID NO 33), mFwe4-Rv 5′-GCGTTCATGATCATCCACAC-3′ (SEQ ID NO 34).

### Cloning

cDNA encoding mFwe isoforms was amplified from total spleen cDNA of adult C57BL/6 mice using the primers: mFwe1 (5′-GCAGCGTTTAGCATGAG-3′ (SEQ ID NO 35), 5′-TCACCCGCAGTAGAAGAC-3′ (SEQ ID NO 36)), mFwe2 (5′-GCAGCGTTTAGCATGAG-3′ (SEQ ID NO 37), 5′-CTCGAAAGTCTCCGCCA-3′ (SEQ ID NO 38)), mFwe3 (5′-GCAGCGTTTAGCATGAG-3′ (SEQ ID NO 39), 5′-AAATGGTGTTTCTGTTCGG-3′ (SEQ ID NO 40)), and mFwe4 (5′-AGCGGCTCGGGCGCCGCCGGA-3′ (SEQ ID NO 41), 5′-CTCGAAAGTCTCCGCCA-3′ (SEQ ID NO 42)). A haemagglutinin (HA) tag sequence was included at the 3′ end of each mFwe cDNA by PCR. The cDNAs were cloned into pUASp vector (DGRC) using BamHI and XbaI restriction sites (for mFwe1-HA and mFwe3-HA), Xba I sites (for mFwe2-HA), or NotI and XbaI sites (for mFwe4-HA). Microinjection of these cDNA constructs into fly embryos was performed according to standard protocols.

### Computer Tomography

Micro-computer tomography analyses were done in the Molecular Imaging Unit of CNIO using eXplore Vista micro PET-CT (GE Healthcare, United Kingdom) and MMWKS Vista-CT 4.7 software following standard procedures.

Transgenic Flies and Clone Induction mFwe isoforms transgene expression in Drosophila, gain-of-function assays and their analyses were performed as described previously (Rhiner et al., ibid).

### Quantifications

Areas of EGFP-marked clones and wing discs were quantified using Adobe Photoshop (Adobe Systems Inc.). Quantification of Ki67-stained sections of papillomas and papilloma-surrounding skin (five mice per genotype) was performed automatically using AxioVision software (Carl Zeiss, Germany). All papillomas per mouse were analyzed. Papilloma-surrounding skin is normal-looking skin that occupies 1000 μm at each side of the tumor. The data represent Ki67-positive area as a percentage of total area measured. For quantification of cell proliferation in wild-type and knock-out mice not treated with DMBA/TPA, Ki67-positive cells were counted manually in 20 photos at 40× magnification per mouse. The data represent number of Ki67-positive cells per μm² measured. Quantification of apoptosis in sections of papillomas and papilloma-surrounding skin stained for activated caspase 3 (three mice per genotype) was performed manually by counting the number of activated caspase 3-positive cells in photos at 40× magnification that comprise all papillomas and 1000 μm of normal skin at each side of every papilloma. The data represent number of activated caspase 3-positive cells per μm² measured.

### Statistical Analyses

Statistical analyses were performed by Student's t, chi, and Mann-Whitney tests using Excel (Microsoft Office) or GraphPad Prism (GraphPad Software). For tumor-free curves, the log-rank test was used. Data represent means±s.e.m.

### Example 1

### mFwe Isoform Expression in Adult Mouse Tissues

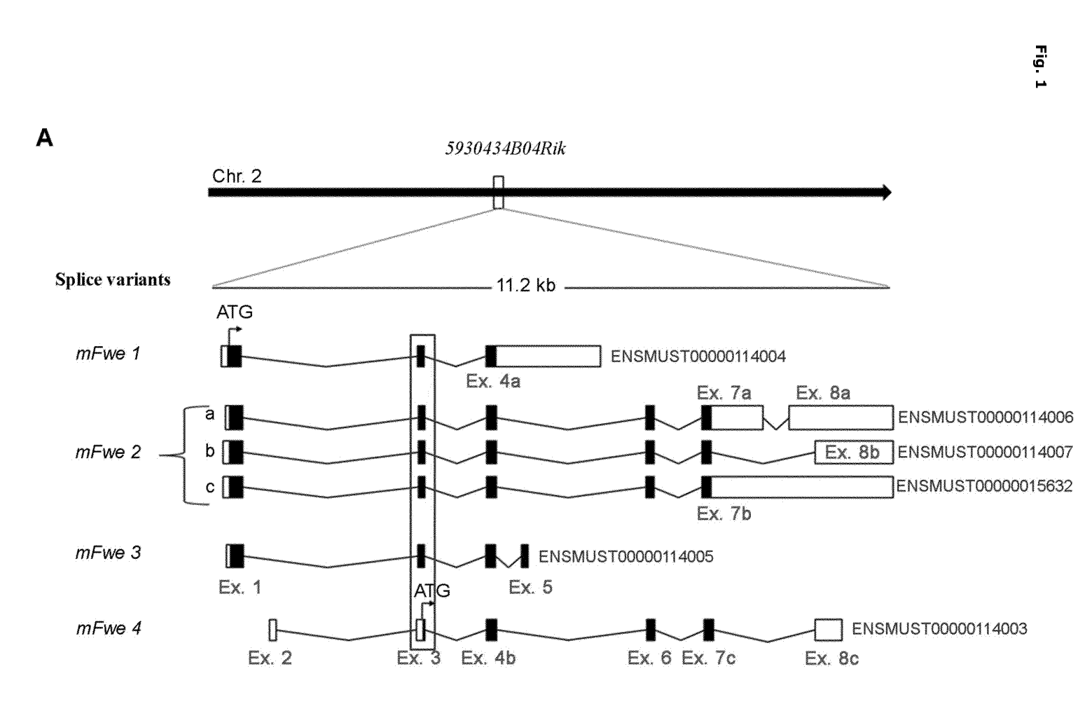

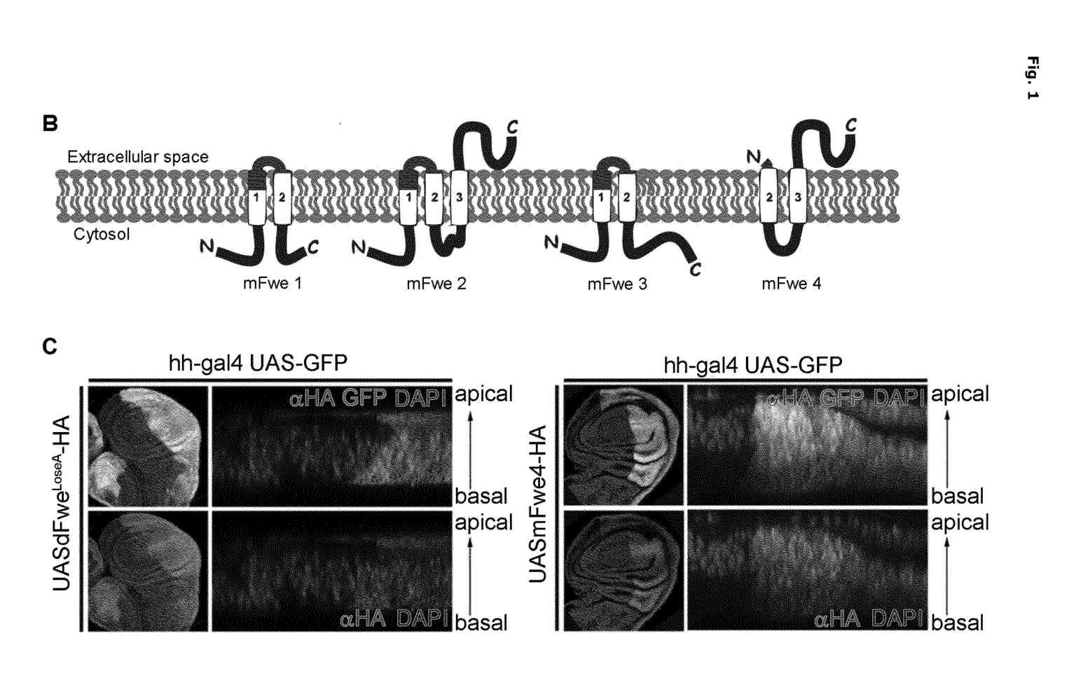

Drosophila Flower belongs to a unique superfamily of small proteins called CG6151-P, which are conserved from fungi to humans. All homologues share the putative conserved protein domain CG6151-P (Marchler-Bauer et al., (2009), Nucl Acids Res 37, D205-10). Except for dFwe, the function of the remaining homologues is unknown. 5930434B04Rik is the single predicted homologue of dFwe sharing 35% identity at the protein sequence level. The 5930434804Rik locus produces six alternatively spliced protein coding transcripts (FIG. 1A). These encode four protein isoforms, which we named mFwe 1, mFwe 2, mFwe 3 and mFwe 4, all predicted to be membrane proteins (FIG. 1B). The four isoforms differ in the number of transmembrane domains and in their C- or N-terminal domains.

To analyze the expression of the mFwe splice variants in various tissues of adult C57BL/6 mice, we performed real-time quantitative PCR. We grouped mFwe mRNA splice variants into four different classes—mFwe1, mFwe2 (mFwe2a, mFwe2b, mFwe2c), mFwe3, and mFwe4—because these different coding sequences generate four mFwe protein isoforms (FIG. 1A, B). The average expression level of these transcripts in several organs of adult wild-type mice is low, with mFwe 1 and mFwe 2 being the most abundant of all (FIG. 5 A-E). The higher expression of mFwe transcripts in tissues such as eyes and brain (FIG. 5 A-D), as compared to their abundance in the rest of the tissues analyzed, is consistent with the described expression and function of dFwe in the Drosophila nervous system.

### Example 2

### Analysis of mFwe Isoforms by Gain-of-Function Assays in Drosophila

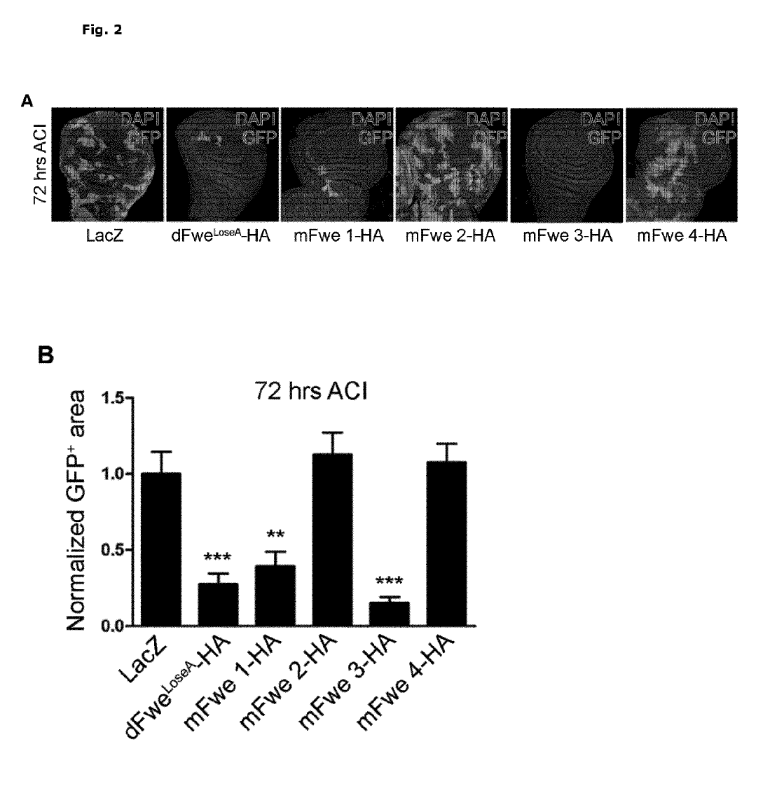

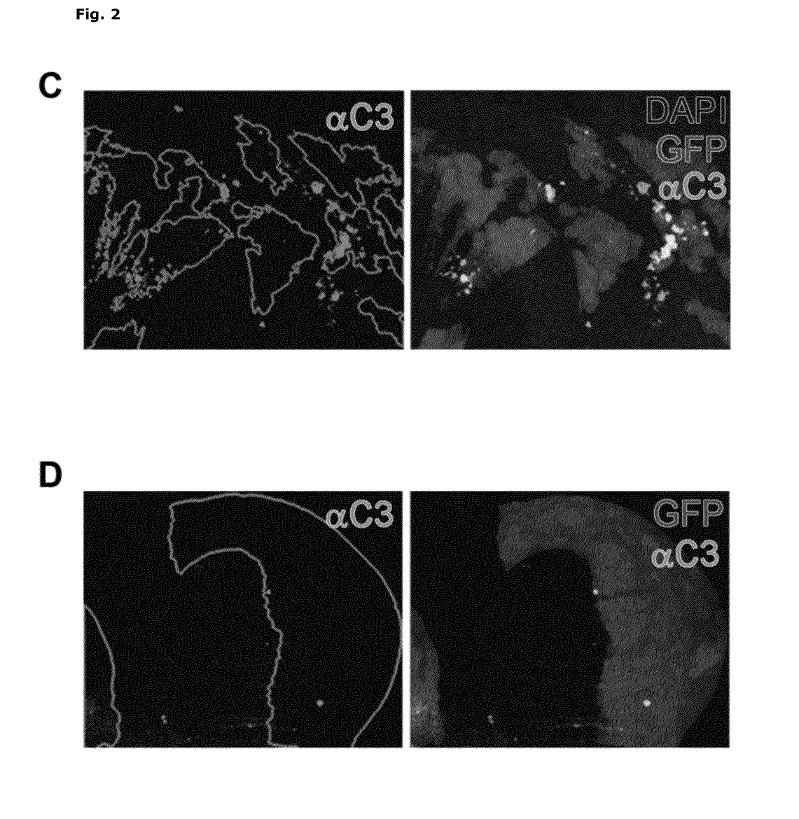

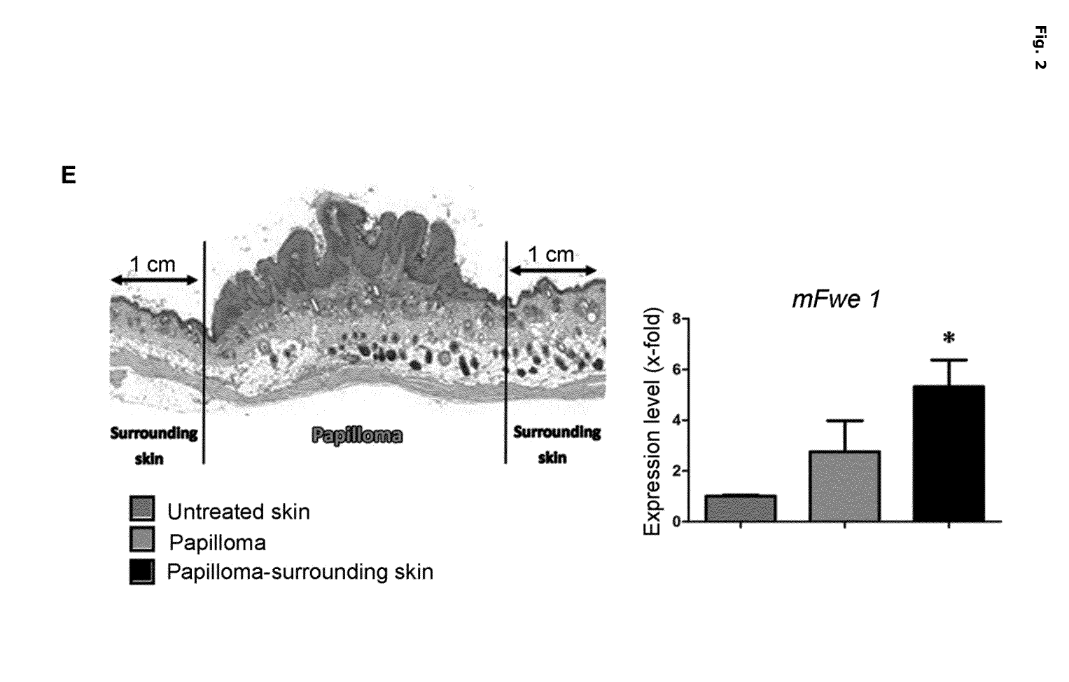

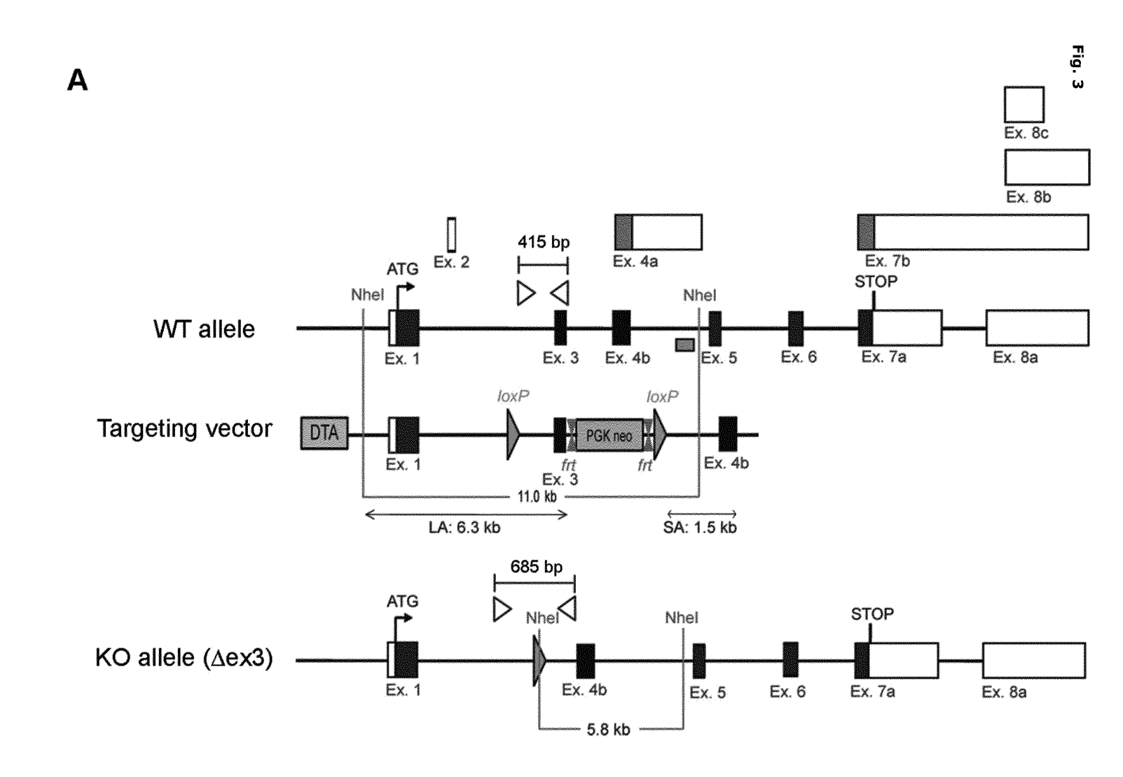

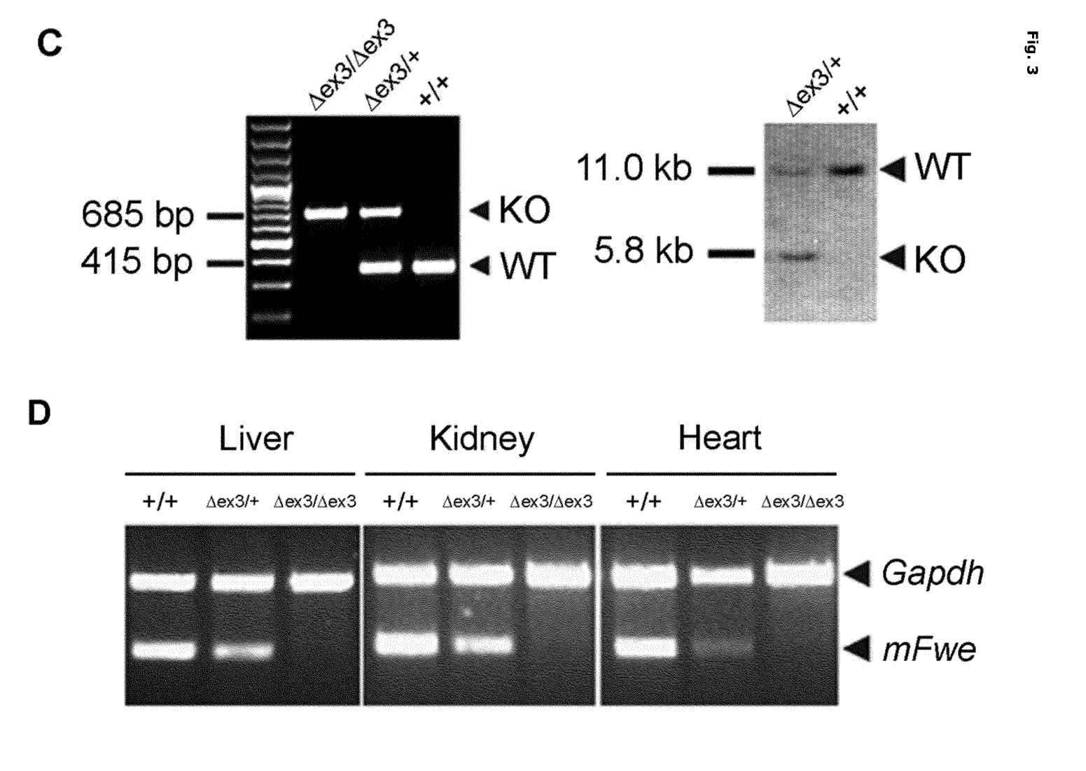

To find out whether mFwe protein isoforms could have a function similar to that of dFwe(LoseA/B) proteins, we tested the effect of their overexpression on cell survival. A previous study showed that overexpression of dFwe(LoseA/B) in Drosophila S2 cultured cells or in clones of cells in Drosophila larvae epithelia induced cell death and clone disappearance (Rhiner et al., ibid). When we overexpressed mFwe isoforms in several types of mammalian cells in culture, we did not observe a similar effect (data not shown). Thus, we assayed the function of mFwe isoforms by expressing them as transgenes in Drosophila (FIG. 1C). We observed that overexpression of HA-tagged mFwe 1 and mFwe 3 (mFwe 1-HA and mFwe 3-HA, respectively) in clones of cells in Drosophila wing imaginal discs over time induced apoptosis and reduced clone survival to a similar extent as the overexpression of dFwe(LoseA)-HA did (FIG. 2A-C, FIG. 6). In contrast, overexpression of mFwe 2-HA and mFwe 4-HA in clones, did not affect clone survival, similar to the overexpression of the LacZ control (FIG. 2A, B and FIG. 6). The reduced clone survival upon overexpression of mFwe 1-HA and mFwe 3-HA was not due to a toxic effect of heterologous protein overexpression, because overexpression of mFwe 1-HA or mFwe 3-HA throughout the whole posterior imaginal disc compartment or in the entire fly did not compromise cell viability (FIG. 2D and data not shown). These results suggest that the ability of dFwe(LoseA/B) to induce cell selection by non-autonomous apoptosis could be conserved in mammals.

### Example 3

### mFwe mRNA is Induced in Papilloma-Surrounding Skin

Previous studies in Drosophila showed that the expression of two of the dfwe alternative transcripts, dfwe(LoseA) and dfwe(LoseB), is restricted specifically to cells of lower fitness, for example those cells that surround dMyc overexpressing clones of cells (Rhiner et al., ibid). We reasoned that, similarly, mammalian models of tumorigenesis could provide situations where cells of different fitness levels are confronted within a tissue. Therefore, measuring the expression level of mFwe transcripts in a tumor and the surrounding non-affected tissue could provide information on the fitness status of tumor cells relative to the adjacent normal cells. Thus, we checked whether the mFwe transcripts were differentially expressed in papillomas and surrounding normal tissue after subjecting C57BL/6 mice to the DMBA/TPA carcinogenesis protocol (FIG. 2E). We found that mFwe 1 showed the highest expression level in DMBA/TPA-treated papilloma-surrounding tissue and lowest expression in wild-type skin of age-matched mice that were not treated with DMBA/TPA (FIG. 2E). We observed a similar pattern of expression for mFwe 2 (FIG. 5F). Taken together, the study of mFwe isoforms overexpression in Drosophila (FIG. 2A-D) and the expression pattern of mFwe isoforms in skin papillomas and papilloma-surrounding skin (FIG. 2E, FIG. 5F) suggest that, as dFwe(LoseA/B), mFwe 1 marks cells as “losers”.

### Example 4

### mFwe Knock-Out Mice

To further study the possible function of mFwe as a marker of potentially less fit cells in vivo, we generated mFwe knock-out mice by targeted deletion of exon 3, which affects all isoforms (FIG. 3A-C). Hereafter, we designate the mFwe targeted allele Dex3 to specify the deletion of mFwe exon 3 and we refer to the mice carrying this allele as mFwe knock-out mice. After Cre-mediated excision of the loxP-flanked exon 3 (FIG. 3A), a frameshift causes mRNA splicing to occur between exon 1 and exon 4, thus generating a pre-mature stop codon at the beginning of exon 4 (FIG. 7A). The resulting truncated protein, encoded by exon 1, is 41 amino acids long and is predicted to be a soluble protein (FIG. 7A). Since it is not exposed to the cell surface, we presume that it does not have any function.

We verified the absence of mFwe mRNA expression in mFwe(Dex3/Dex3) mice and the reduced mFwe mRNA expression in mFwe heterozygous mice (FIG. 3D). The deletion of exon 3 and the generation of a premature stop codon were confirmed by sequencing the corresponding mFwe transcripts in mFwe(Dex3/Dex3) mice (FIG. 7A). We did not detect expression of the remaining short transcript upon transfection in cultured cells, suggesting that the truncated mFwe protein is non-functional and is rapidly degraded within the cell (data not shown).

### Example 5

### mFwe-Deficient Mice Show a Normal Phenotype and are Protected Against Skin Carcinogenesis

mFwe-deficient mice develop and grow normally (FIG. 7B-E) unlike dfwe mutants (Rhiner et al., ibid), which are not viable. Anatomical and histological examinations did not reveal any abnormality in the mFwe-deficient animals when compared to the mFwe heterozygous or wild-type littermates (FIG. 7B-E).

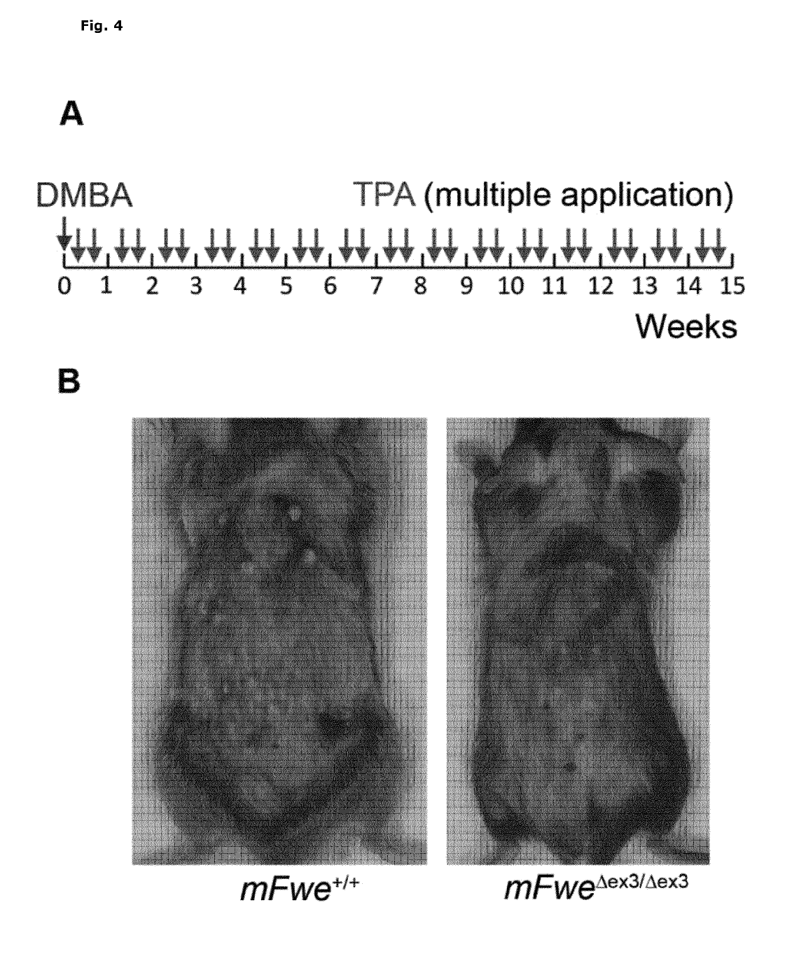

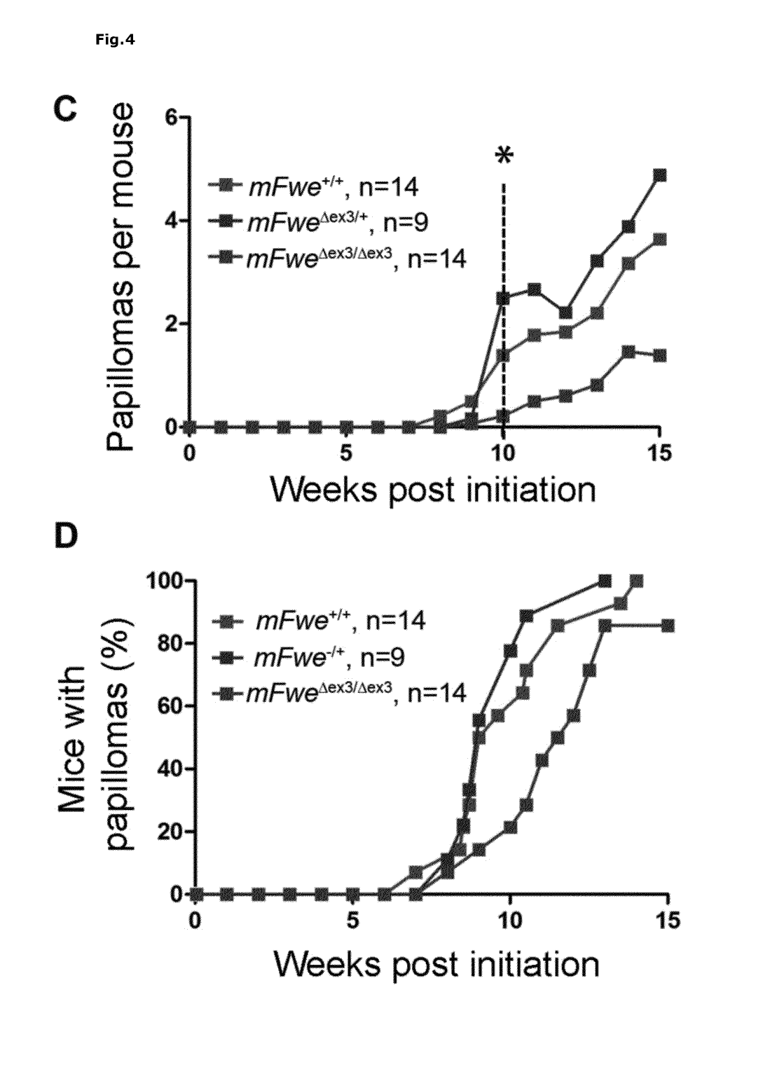

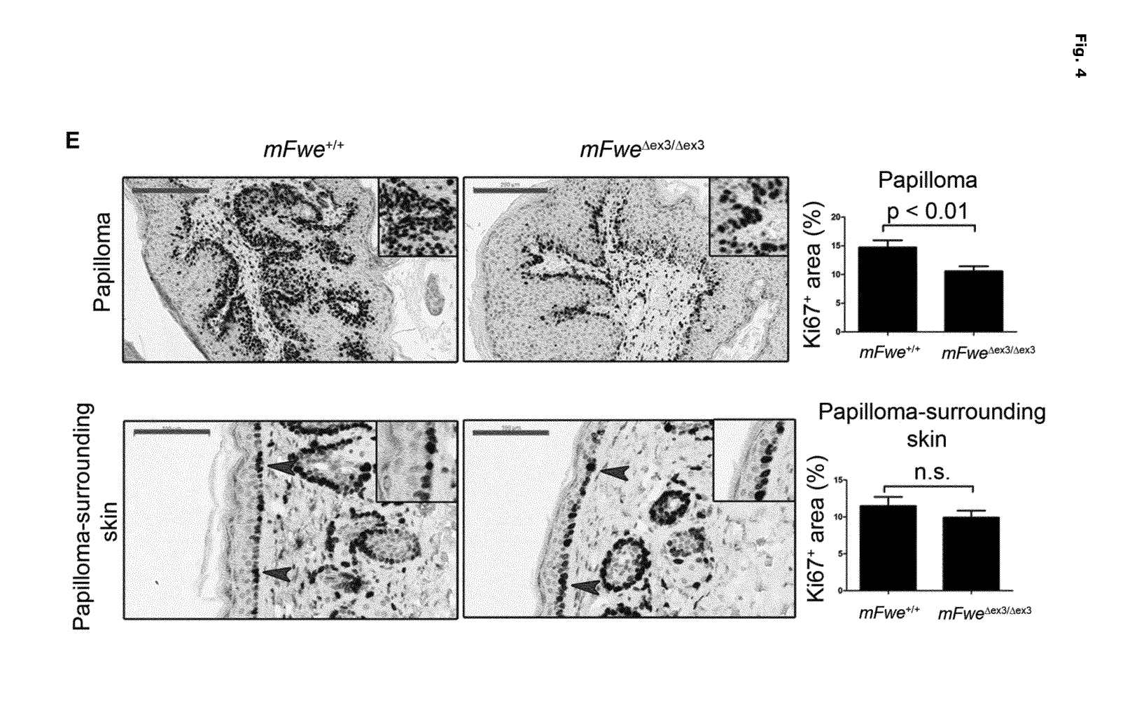

Since reduction of dFwe expression can slow down the expansion of dMyc-overexpressing pretumoral clones of cells without affecting normal tissue growth, we sought to test whether lack of mFwe could have a beneficial effect on tumorigenesis in mice. Therefore, we subjected constitutive mFwe knock-out, mFwe heterozygous and wild-type mice to the DMBA/TPA skin carcinogenesis protocol, which induces skin papilloma formation. (FIG. 4A). This protocol entails a single treatment with a low dose of the 7,12-dimethylbenzanthracene (DMBA) carcinogen, which “initiates” pretumoral lesions in the epidermis by causing oncogenic mutations in the ras gene (Quintanilla et al., (1986), Nature 322, 78-80), and subsequent repeated treatments with the tumor promoter 12-O-tetradecanoylphorbol-13-acetate (TPA), which promotes papilloma formation by stimulating the proliferation and clonal expansion of initiated (mutant) cells ((diGiovanni, (1992), Pharmac Ther 54, 63-128); Abel et al., (2009) Nat Protoc 4, 1350-62; Yuspa, (1998), J Dermatol Sci 17. 1-7)). We were particularly interested in analyzing the initiation and promotion phases of tumorigenesis because they represent the early, pre-neoplastic stages of skin carcinogenesis (FIG. 4A).

The number of papillomas that appeared in mFwe-deficient mice was strongly reduced (1.5 papillomas/mouse at week 15) compared to the number of papillomas observed in wild-type (3.9 papillomas/mouse at week 15) or mFwe heterozygous (5 papillomas/mouse at week 15) littermates (FIG. 4 B, C). This difference remained significant from week 10 until the end of the DMBA/TPA carcinogenesis protocol (FIG. 4A-C). Moreover, three out of fourteen mFwe(Dex3/Dex3) mice did not develop any tumors during the entire protocol. All mFwe(Dex3/Dex3) mice started to develop papillomas at slightly later time-points (FIG. 4D), suggesting that the absence of mFwe protein was also delaying the process of papilloma formation in the skin.

### Example 6

### Decreased Proliferation of mFwe-Deficient Papilloma Cells

To understand how deficiency of mFwe could account for the reduced number of skin papillomas induced by DMBA/TPA, we analyzed papillomas and papilloma-surrounding skin both macroscopically and at the tissue/cell level.

Examination of tumor samples according to previous classification criteria (Klein-Szanto, (1997), Pathology of Neoplasia and Preneoplasia in Rodents, Vol. 2. pp. 1-18) and staining for the keratinocyte differentiation marker cytokeratin 10, revealed that papillomas from the three experimental groups were of similar size and consisted of well-differentiated, hyperplastic lesions with no atypical cells, or with very few atypical cells in the basal layer (FIG. 8 A, B).

To evaluate whether the reduced number of papillomas observed in the mFwe-deficient mice could reflect compromised proliferation of mFwe-deficient pretumoral cells, we stained papillomas and papilloma-surrounding tissue of wild-type and mFwe-deficient mice with Ki67, a marker of proliferating cells (FIG. 4E). Interestingly, the level of cell proliferation was significantly higher (p<0.01, t test) in wild-type papillomas than in papillomas of mFwe-deficient mice. In contrast, the proliferation level in the papilloma-surrounding skin was similar in wild-type and mFwe knock-out mice (FIG. 4E). Furthermore, we did not detect any significant difference in cell proliferation between wild-type and mFwe knock-out epidermis, which was not treated with DMBA/TPA (FIG. 8C). Thus, the lower cell proliferation observed in papillomas of mFwe-deficient mice compared to wild-type mice (FIG. 4E) is not due to an intrinsic, reduced capacity of mFwe-deficient skin cells to proliferate and is therefore specifically affected in mFwe-deficient skin papilloma cells. Thus, the lower number of papillomas in mFwe-deficient mice could be due, at least in part, to the reduced capacity of skin papilloma cells to proliferate in the absence of mFwe.

In Drosophila, increased expression of dFwe(LoseA/B)triggers cell non-autonomous apoptosis (Rhiner et al., 2010). To check whether expression of mFwe affects apoptosis levels in papilloma-surrounding skin relative to papillomas, we measured the number of activated caspase 3-positive cells in papillomas and papilloma-surrounding skin of wild-type and mFwe(Dex3/Dex3) mice (FIG. 8 D-E). For both wild-type and mFwe(Dex3/Dex3) mice, we observed a higher number of apoptotic cells, 0.006 cells/mm² and 0.0016 cells/mm² respectively, in papilloma-surrounding skin compared to papillomas (FIG. 8E). We also observed that mFwe(Dex3/Dex3) mice showed an increased number of apoptotic cells in both papillomas and papilloma-surrounding skin compared to wild-type mice. We expressed the difference in apoptosis levels between papilloma-surrounding skin and papillomas as a ratio of the number of apoptotic cells in papilloma-surrounding skin and the number of apoptotic cells in papillomas (FIG. 8E). By comparing the ratios obtained for both genotypes, we could estimate to what extent the levels of apoptosis in a papilloma and in the adjacent skin differ, i.e. the relative levels of apoptosis. The ratio obtained for mFwe(Dex3/Dex3) mice showed that for each cell dying inside a papilloma there are 1.62 cells dying in the adjacent tissue, whereas in wild-type mice, for each cell dying inside a papilloma, there are 1.74 cells dying outside it. The slightly smaller difference in apoptosis levels (1.62) between papilloma and papilloma-surrounding skin in the mFwe mutants suggests that, somehow, mFwe expression is needed for papillomas to grow by increasing apoptosis of the surrounding normal cells.

The examples of the present description show for the first time data about the possible function of mouse Flower (mFwe)—the predicted homologue of the Drosophila cell competition gene dFlower. Like dFwe(LoseA/B) isoforms, mFwe 1 and mFwe 3 induce non-autonomous apoptosis when over-expressed in Drosophila wing imaginal disc cells: apoptosis was only observed when these proteins were overexpressed in clones of cells in the epithelial tissue, whereas no cell death was triggered if the entire tissue overexpressed mFwe1 or mFwe3 (FIG. 2A-D). These results suggest a functional conservation between mFwe1/3 and dFwe(LoseA/B).

In Drosophila, cells of higher fitness use the Flower code to proliferate by inducing expression of dFwe(LoseA/B) in the surrounding loser cells (Rhiner et al., 2010). Similarly, we observe higher expression of certain mFwe isoforms, mainly in papilloma-surrounding skin compared to papillomas (FIG. 2E). This finding again indicates a possible functional conservation between mFwe and dFwe and further suggests that cellular selection based on relative fitness states could drive the clonal expansion of pretumoral cells at the expense of surrounding wild-type cells.

Here, we find that during skin papilloma formation mFwe 1 and mFwe 2 isoforms increase significantly their expression (FIGS. 2E, S1F); however in Drosophila only mFwe 1 overexpression is able to mark cells as “losers” (FIG. 2A,B). Similarly, mFwe 3 and mFwe 4 tend to increase their expression during skin papilloma formation (FIG. 5F); however, overexpression of mFwe 3, but not of mFwe 4, labels cells as “losers” in Drosophila (FIG. 2A,B). Taken together, we suggest that the function of mFwe during skin papilloma formation can be based on a molecular code that relies simply on the overexpression of the mouse “Lose”-like isoforms (mFwe 1 and to a lesser extent mFwe 3). Likewise, overexpression of dFweLose isoforms in Drosophila wing imaginal discs is sufficient and necessary to label cells as “losers”. Thus, in mice “healthy” cells seem to express nothing similar to dFwe(ubi), but “loser” cells express mFwe 1 isoform that behaves as dFwe(Lose) does.

Another indication of a functional conservation between these proteins is our finding that mFwe deficiency reduces the capacity of skin papilloma cells to proliferate (FIG. 4E, FIG. 8C). The slower growth of a clone of pretumoral cells could partially explain why mFwe-deficient mice develop a lower number of papillomas when treated with DMBA/TPA (FIG. 4C). Importantly, this growth disadvantage occurs only in skin papilloma cells and does not affect mouse development or organ size (7B-E).

In addition, we report that papilloma-surrounding skin, where the “Lose”-like mFwe 1 isoform is upregulated, shows increased number of apoptotic cells as compared to papillomas in both wild-type and mFwe(Δex3/Δex3) mice (FIG. 8D-E). However, the difference in apoptosis levels between a papilloma and papilloma-surrounding skin is slightly reduced in mFwe(Δex3/Δex3) mice, suggesting that expression of mFwe 1 could be the cause for the increase of apoptosis in papilloma-surrounding skin relative to a papilloma. At present, we do not know why mFwe mutant mice have an elevated number of apoptotic cells in both papillomas and papilloma-surrounding skin compared to wild-type mice (FIG. 8E). Further studies are needed to clarify the relationship between apoptosis, mFwe expression and cell selection.

In summary, we provide evidence that mFwe deficiency specifically impairs skin papilloma formation and proliferation, without affecting normal tissue growth.

Human epithelial cancers originate as a result of successive accumulation of genetic alterations in the tissue. Clonal expansion of mutant cells is necessary for the fixation of additional mutations and subsequent tumor formation. It is proposed that an active process of cell selection determines which cell persists in a tissue and forms a tumor. Such cell selection is based on a cell's fitness status, where a mutant cell of higher fitness can proliferate at the expense of cells of lower fitness, such as normal cells or cells carrying other types of mutations. The process of clonal expansion of mutant cells that causes no visible morphological change in the tissue is referred to as “field cancerization”. This term was first used in the clinic to explain the appearance of multiple primary tumors in the same region of a tissue or the local recurrence of secondary tumors following surgical resection. Field cancerization precedes tumor formation and is reported to occur in a wide variety of epithelial cancers. The present application provides means and methods to detect such cancer cells before a tumor is formed by facilitating biomarkers of cancerization fields. By applying antibodies or similar ligands for detecting and binding to the pretumor cell clones, the formation of cancer fields may be detected and inhibited, so that a tumor never forms. Cancer treatment at the earliest possible stage is the best option, but such therapy is precluded The present invention thereby facilitates the detection and therapy of early pretumoral stages of cancer.

### Example 7

### Analysis of the Expression of FLOWER Isoforms in Human Tumors and Tumor Stroma Samples

The tumor samples of human origin were procured from Dr. Davide Soldini, Zurich University Hospital, and Ohio State University Medical Centre, James Cancer Center. Briefly 4 lung tumor samples, 1 breast tumor sample, 1 colon tumor sample, 1 urinary bladder tumor sample and 2 head and neck tumor samples along with the respective host tissue from tumor boundary and healthy tissue samples from the respective patients were analyzed for the expression of the above mentioned flower isoforms using qPCR. The qPCR was conducted using a custom made, commercially available kit from Invitrogen catalog-#-4331182 which performs qPCR for the cDNA sequences 1) NM_{—}001135775.2, 2) NM_{—}001242369.1, 3) NM_{—}001242370.1 and 4) NM_{—}017586.3 representing the cDNA sequences coded by the transcripts ENST00000291722, ENST00000540581, ENST00000542192 and ENST00000316948 respectively. The qPCR was conducted as per the manufacturer's protocol in the total RNA isolated from A) the tumor tissue B) the tumor boundary tissue and C) the healthy tissue from the respective patient. FIG. 9 shows the zones from and around the tumor from where the tissue samples were collected. The qPCR results of the 9 tumor samples, tumor host tissue and healthy tissue from the respective patients is represented in FIG. 10. The results of the qPCR experiment suggest that expression of all the four transcripts of flower gene ENST00000316948, ENST00000291722, ENST00000540581 and ENST00000542192 was low in the healthy tissue samples from all the 9 patients. Further we identified a unique pattern of the expression of these transcripts in the tumor tissue and in the host tissue surrounding the tumors. We identified that either one or both of the transcripts ENST00000291722 and ENST00000542192 were consistently over expressed in the host tissue surrounding the tumors. On the other hand we observed that either one or both of the transcripts ENST00000316948 and ENST00000540581 were overexpressed in the tumor tissue of all the 9 tumor samples of different origin. Based on this data we infer that the over-expression of either ENST00000291722 or ENST00000542192 or both within the tumor host tissue and the over-expression of ENST00000316948 or ENST00000540581 or both within the tumor tissue can serve as potential biomarkers for identification and characterization of cancerous zones.

### Example 8

### Characterization of FLOWER Isoforms as FLOWER(UBI) and FLOWER(LOSE) in Human Cancer Cell Lines

The 4 different isoforms of Flower gene represented by the 4 transcript sequences ENST00000316948, ENST00000291722, ENST00000540581 and ENST00000542192 were characterized as the FLOWER(UBI) and FLOWER(LOSE) isoforms in MCF-7 and HCT human cancer cell lines of breast and colon origin. We hypothesized that in a co-culture experiment (2 cell lines at one time) of cancer cells which exclusively expresses one particular flower isoform, the cell lines which shows higher apoptotic fraction will behave as LOSE and the cell line which shows low apoptotic fractions will behave as UBI (kindly refer to the scheme of the research strategy (FIG. 11)).

The flower gene was knocked out of the genome of the MCF-7 and HCT cancer cell lines using zinc finger nucleases. To validate the successful knockout of Flower gene from these cell lines the expression of the Flower mRNA and Flower protein was observed using qPCR and western blotting respectively (FIG. 12). The results show no expression of either flower mRNA or protein in MCF-7 and HCT cells. After the successful creation of the knockout cell lines we synthesized cDNA vectors of the 4 flower isoforms under SV40 promoter. We then individually transfected the Flower (−/−) cells transfected the MCF-7 and HCT cells with one flower isoform per culture dish to generate four different cell lines of MCF-7 origin and four cell lines of HCT origin which exclusively express ENST00000316948, ENST00000291722, ENST00000540581 or ENST00000542192. Thus the 8 cell lines generated were

| | |
|---|---|
| MCF-7(ENST00000316948) | HCT(ENST00000316948) |
| MCF-7(ENST00000291722) | HCT(ENST00000291722) |
| MCF-7(ENST00000540581) | HCT(ENST00000540581) |
| MCF-7(ENST00000542192) | HCT(ENST00000542192) |

The 8 cell lines were co-transfected with CFP and GFP to generate 16 different cell lines 8 of MCF-7 origin and 8 of HCT origin (FIG. 13). Now all the cell lines were co-cultured as using cell culture protocol as described previously by (Gogna et al., 2012 J. Biol. Chem. 287, 2907-2914; Madan et al., 2012 Biochemical J. 443, 811-820) in a scheme represented in table (FIG. 13). Next after co-culturing the cells for 24 h, the cells were stained for annexin-V as per the protocol provided by Gogna et al and then we used the BD FACS ARIA cell sorting technology to sort the GFP+ and CFP+ cells. This way we separated the two cell lines expressing the two different isoforms of the flower gene. Post cell sorting the cells were analyzed for the annexin-V to identify if either of the cell lines expressing individual flower isoforms might induce an apoptotic cell death in the co-cultured cells. Based on this experimental design we received consistent results for the apoptotic fractions of the cell lines expressing individual flower isoforms in both MCF-7 and HCT cell lines (FIG. 14). According to the results obtained via this technique we observed that the MCF-7 and HCT cells expressing flower isoforms ENST00000316948 and ENST00000540581 induced a significant increase in the apoptotic fraction of MCF-7 and HCT cells expressing flower isoforms ENST00000291722 or ENST00000542192 (FIG. 14). Interestingly co-culture of MCF-7 and HCT cells expressing ENST00000316948 and ENST00000540581 isoforms did not show any significant change in the apoptotic fractions. Similarly co-culture of MCF-7 and HCT cells expressing ENST00000291722 or ENST00000542192 isoforms did not show any significant change in the apoptotic fractions. This data shows that the flower isoforms ENST00000316948 and ENST00000540581 function as Flower(UBI) isoforms and are expressed in the tumor regions of a variety of cancer samples. Further flower isoforms ENST00000291722 or ENST00000542192 function as Flower(LOSE) and are expressed at the interface of tumor and the healthy tissue (tumor boundaries).

1. A method for diagnosing cancer or a pre-cancerous state in a human subject, comprising:
a) determining in a biological sample obtained from said human subject
i. the presence, location, and/or quantity of a nucleic acid sequence identified by one of SEQ ID NO 1, 2, 3, 4, 5, 6 or 13, or
ii. the presence, location, and/or quantity of a protein identified by one of SEQ ID NO 7, 8, 9, 10, 11, 12, 14, 15, 16 and/or 17, and
b) comparing said presence, location, and/or quantity of said nucleic acid or said protein to a standard.

2. A method according to claim 1, characterized in that
a. said presence, location, and/or quantity of a nucleic acid sequence is determined by RT-PCR or FISH, or
b. said presence, location, and/or quantity of a protein is determined by Western Blot or immunofluorescence.

3. A ligand capable of selectively binding to a protein identified by one of SEQ ID NO 7, 8, 9, 10, 11, 12, 14, 15, 16 and/or 17, wherein the ligand is optionally covalently attached to a detectable label.

4. A ligand according to claim 3, wherein the ligand is a human or humanized immunoglobulin or a fragment thereof.

5. A ligand according to claim 3, wherein the ligand is an antibody or antibody fragment.

6. A method for detecting cancer or a precancerous state in a patient, comprising detecting the cancer or precancerous state with the ligand of claim 3.

7. A nucleic acid molecule capable of hybridizing to a nucleic acid sequence identified by one of SEQ ID NO 1, 2, 3, 4, 5, 6 or 13, wherein the nucleic acid molecule is optionally covalently attached to a detectable label.

8. A method for detecting cancer or a precancerous state in a patient, comprising detecting the cancer or precancerous state with the nucleic acid molecule of claim 7.

9. A method for preventing or treating a disease, particularly neoplastic disease, more particularly carcinoma, comprising administering the ligand of claim 3 to a patient in need thereof.

10. The method according to claim 9, wherein the ligand is an antibody or antibody fragment.

11. The method according to claim 9, wherein the ligand is a human or humanized immunoglobulin, particularly immunoglobulin gamma, or a fragment thereof.

12. The method according to claim 9, wherein the ligand is covalently attached to a radioisotope or toxin.

13. A method for preventing or treating a disease, particularly neoplastic disease, more particularly carcinoma, comprising administering an inhibiting nucleic acid molecule comprising the nucleic acid molecule of claim 7, or an inhibiting nucleic acid molecule capable of hybridizing to an operon expressing one of SEQ ID NO 1, 2, 3, 4, 5, or 6.

14. An expressed nucleic acid molecule encoding an inhibiting nucleic acid molecule according to claim 13 under control of a promoter sequence operable in a human cell, for prevention or therapy of disease.

15.-18. (canceled)


SEQUENCE LISTING (44 sequences)

SEQ ID NO: 1 - DNA, 519 bp, Mus musculus
atgagcagctcaggtggggcgcccggggcgtccgccagctctgcgccgcccgcgcaggaagagggcatgacgtggtggtaccgctggctgtgtcgcctgtctggggtgctgggggcagtctcttgcgcgatctctggcctcttcaactgcatcaccatccaccctctgaacattgcggccggcgtgtggatgatcatgaatgccttcatcttgttgctgtgtgaggcgcccttctgctgccagttcatcgagtttgcaaacacagtggcggagaaggtggaccggctgcgctcctggcagaaggctgtcttctactgcgggatggcggtcgttcccatcgtcatcagcctgaccctgaccacgctgctgggcaacgccatcgcctttgctacgggggtgctgtacggactctctgctctgggcaaaaagggcgatgcgatctcctatgccaggatccagcagcagaggcagcaggcggatgaggagaagctcgcggagaccctggagggggagctgtga

SEQ ID NO: 2 - DNA, 393 bp, Mus musculus
atgagcagctcaggtggggcgcccggggcgtccgccagctctgcgccgcccgcgcaggaagagggcatgacgtggtggtaccgctggctgtgtcgcctgtctggggtgctgggggcagtctcttgcgcgatctctggcctcttcaactgcatcaccatccaccctctgaacattgcggccggcgtgtggatgatgatggcggtcgttcccatcgtcatcagcctgaccctgaccacgctgctgggcaacgccatcgcctttgctacgggggtgctgtacggactctctgctctgggcaaaaagggcgatgcgatctcctatgccaggatccagcagcagaggcagcaggcggatgaggagaagctcgcggagaccctggagggggagctgtga

SEQ ID NO: 3 - DNA, 344 bp, Mus musculus
atgctgactgcagcaggaagcacagccgagctgtcagcttgcgcgatctctggcctcttcaactgcatcaccatccaccctctgaacattgcggccggcgtgtggatgatcatgaatgccttcatcttgttgctgtgtgaggcgcccttctgctgccagttcatcgagtttgcaaacacagtggcggagaaggtggaccggctgcgctcctggcagaaggctgtcttctactgcgggatggcggtcgttcccatcgtcatcagcctgaccctgaccacgctgctgggcaacgccatcgcctttgctacgggggtgctgtacggactctctgctctgggcaaaaa

SEQ ID NO: 4 - DNA, 489 bp, Mus musculus
atgagcagctctgcgccgcccgcgcaggaagagggcatgacgtggtggtaccgctggctgtgtcgcctgtctggggtgctgggggcagtctcttgcgcgatctctggcctcttcaactgcatcaccatccaccctctgaacattgcggccggcgtgtggatgatcatgaatgccttcatcttgttgctgtgtgaggcgcccttctgctgccagttcatcgagtttgcaaacacagtggcggagaaggtggaccggctgcgctcctggcagaaggctgtcttctactgcgggatggcggtcgttcccatcgtcatcagcctgaccctgaccacgctgctgggcaacgccatcgcctttgctacgggggtgctgtacggactctctgctctgggcaaaaagggcgatgcgatctcctatgccaggatccagcagcagaggcagcaggcggatgaggagaagctcgcggagaccctggagggggagctgtga

SEQ ID NO: 5 - DNA, 702 bp, Mus musculus
atgagcagctcaggtggggcgcccggggcgtccgccagctctgcgccgcccgcgcaggaagagggcatgacgtggtggtaccgctggctgtgtcgcctgtctggggtgctgggggcagtctcttgcgcgatctctggcctcttcaactgcatcaccatccaccctctgaacattgcggccggcgtgtggatgatcatgaatgccttcatcttgttgctgtgtgaggcgcccttctgctgccagttcatcgagtttgcaaacacagtggcggagaaggtggaccggctgcgctcctggcagaaggctgtcttctactgcgggatggcggtcgttcccatcgtcatcagcctgaccctgaccacgctgctgggcaacgccatcgcctttgctacgggggtgctgtacggactctctgctctgggcaaaaaagcccagactgaggctggttccttcgcagcccagcatcccagggagcctgggccattctcagaggggacaagacaggcctttgccaccccagcagttgtctctggggaaatcagaatgcctgcaggggcgatgcgatctcctatgccaggatccagcagcagaggcagcaggcggatgaggagaagctcgcggagaccctggagggggagctgtgaagggctgggcgcccctccctccctgtcccctcttctggctctgtgtgggtccaagtga

SEQ ID NO: 6 - DNA, 576 bp, Mus musculus
atgagcagctcaggtggggcgcccggggcgtccgccagctctgcgccgcccgcgcaggaagagggcatgacgtggtggtaccgctggctgtgtcgcctgtctggggtgctgggggcagtctcttgcgcgatctctggcctcttcaactgcatcaccatccaccctctgaacattgcggccggcgtgtggatgatgatggcggtcgttcccatcgtcatcagcctgaccctgaccacgctgctgggcaacgccatcgcctttgctacgggggtgctgtacggactctctgctctgggcaaaaaagcccagactgaggctggttccttcgcagcccagcatcccagggagcctgggccattctcagaggggacaagacaggcctttgccaccccagcagttgtctctggggaaatcagaatgcctgcaggggcgatgcgatctcctatgccaggatccagcagcagaggcagcaggcggatgaggagaagctcgcggagaccctggagggggagctgtgaagggctgggcgcccctccctccctgtcccctcttctggctctgtgtgggtccaagtga

SEQ ID NO: 7 - protein, 172 aa, Mus musculus
MSSSGGAPGASASSAPPAQEEGMTWWYRWLCRLSGVLGAVSCAISGLFNCITIHPLNIAAGVWMIMNAFILLLCEAPFCCQFIEFANTVAEKVDRLRSWQKAVFYCGMAVVPIVISLTLTTLLGNAIAFATGVLYGLSALGKKGDAISYARIQQQRQQADEEKLAETLEGEL

SEQ ID NO: 8 - protein, 130 aa, Mus musculus
MSSSGGAPGASASSAPPAQEEGMTWWYRWLCRLSGVLGAVSCAISGLFNCITIHPLNIAAGVWMMMAVVPIVISLTLTTLLGNAIAFATGVLYGLSALGKKGDAISYARIQQQRQQADEEKLAETLEGEL

SEQ ID NO: 9 - protein, 114 aa, Mus musculus
MLTAAGSTAELSACAISGLFNCITIHPLNIAAGVWMIMNAFILLLCEAPFCCQFIEFANTVAEKVDRLRSWQKAVFYCGMAVVPIVISLTLTTLLGNAIAFATGVLYGLSALGK

SEQ ID NO: 10 - protein, 162 aa, Mus musculus [sequence omitted]

SEQ ID NO: 11 - protein, 233 aa, Mus musculus
MSSSGGAPGASASSAPPAQEEGMTWWYRWLCRLSGVLGAVSCAISGLFNCITIHPLNIAAGVWMIMNAFILLLCEAPFCCQFIEFANTVAEKVDRLRSWQKAVFYCGMAVVPIVISLTLTTLLGNAIAFATGVLYGLSALGKKAQTEAGSFAAQHPREPGPFSEGTRQAFATPAVVSGEIRMPAGAMRSPMPGSSSRGSRRMRRSSRRPWRGSCEGLGAPPSLSPLLALCGSK

SEQ ID NO: 12 - protein, 191 aa, Mus musculus
MSSSGGAPGASASSAPPAQEEGMTWWYRWLCRLSGVLGAVSCAISGLFNCITIHPLNIAAGVWMMMAVVPIVISLTLTTLLGNAIAFATGVLYGLSALGKKAQTEAGSFAAQHPREPGPFSEGTRQAFATPAVVSGEIRMPAGAMRSPMPGSSSRGSRRMRRSSRRPWRGSCEGLGAPPSLSPLLALCGSK

SEQ ID NO: 13 - DNA, 320 bp, Mus musculus
atgagcagctcaggtggggcgcccggggcgtccgccagctctgcgccgcccgcgcaggaagagggcatgacgtggtggtaccgctggctgtgtcgcctgtctggggtgctgggggcagtctcttgcgcgatctctggcctcttcaactgcatcaccatccaccctctgaacattgcggccggcgtgtggatgatcatgaatgccttcatcttgttgctgtgtgaggcgcccttctgctgccagttcatcgagtttgcaaacacagtggcggagaaggtggaccggctgcgctcctggcagaaggctgtcttctactgcgg

SEQ ID NO: 14 - protein, 49 aa, Mus musculus
GNAIAFATGVLYGLSALGKKGDAISYARIQQQRQQADEEKLAETLEGEL

SEQ ID NO: 15 - protein, 10 aa, Mus musculus
ITIHPLNIAA

SEQ ID NO: 16 - protein, 109 aa, Mus musculus
NAIAFATGVLYGLSALGKKAQTEAGSFAAQHPREPGPFSEGTRQAFATPAVVSGEIRMPAGAMRSPMPGSSSRGSRRMRRSSRRPWRGSCEGLGAPPSLSPLLALCGSK

SEQ ID NO: 17 - protein, 12 aa, Mus musculus
IHPLNIAAGVWM

SEQ ID NO: 18 - DNA, 20 bp, Mus musculus
ctaactacccaagcatcctg

SEQ ID NO: 19 - DNA, 20 bp, Mus musculus
cgcagttgaagagtccagag

SEQ ID NO: 20 - DNA, 20 bp, Mus musculus
tacaccaaagaatgacccac

SEQ ID NO: 21 - DNA, 20 bp, Mus musculus
ctcttcaactgcgtcactat

SEQ ID NO: 22 - DNA, 20 bp, Mus musculus
tgcccactgctatcaaataa

SEQ ID NO: 23 - DNA, 20 bp, Mus musculus
gtatgtcgtggagtctactg

SEQ ID NO: 24 - DNA, 20 bp, Mus musculus
tcatcatacttggcaggttt

SEQ ID NO: 25 - DNA, 20 bp, Mus musculus
gtaacccgttgaaccccatt

SEQ ID NO: 26 - DNA, 20 bp, Mus musculus
ccatccaatcggtagtagcg

SEQ ID NO: 27 - DNA, 20 bp, Mus musculus
tccacacttctctggttctg

SEQ ID NO: 28 - DNA, 20 bp, Mus musculus
gtgagtactgctgtctagcc

SEQ ID NO: 29 - DNA, 20 bp, Mus musculus
cgatgccatttcttatgctc

SEQ ID NO: 30 - DNA, 20 bp, Mus musculus
tgacactcagtcttctccag

SEQ ID NO: 31 - DNA, 21 bp, Mus musculus
caaacacagtagctgagaagg

SEQ ID NO: 32 - DNA, 21 bp, Mus musculus
tagagggaaatggtgtttctg

SEQ ID NO: 33 - DNA, 21 bp, Mus musculus
gtttgctaaatcctgggtgtc

SEQ ID NO: 34 - DNA, 20 bp, Mus musculus
gcgttcatgatcatccacac

SEQ ID NO: 35 - DNA, 17 bp, Mus musculus
gcagcgtttagcatgag

SEQ ID NO: 36 - DNA, 18 bp, Mus musculus
tcacccgcagtagaagac

SEQ ID NO: 37 - DNA, 17 bp, Mus musculus
gcagcgtttagcatgag

SEQ ID NO: 38 - DNA, 17 bp, Mus musculus
ctcgaaagtctccgcca

SEQ ID NO: 39 - DNA, 17 bp, Mus musculus
gcagcgtttagcatgag

SEQ ID NO: 40 - DNA, 19 bp, Mus musculus
aaatggtgtttctgttcgg

SEQ ID NO: 41 - DNA, 21 bp, Mus musculus
agcggctcgggcgccgccgga

SEQ ID NO: 42 - DNA, 17 bp, Mus musculus
ctcgaaagtctccgcca

SEQ ID NO: 43 - DNA, 225 bp, mus musculus
atgagcggctcgggcgccgccggagcggccgcgggcccagccccgccggcacaggaagagggcatgacttggtggtaccgctggctgtgccgcctggcgggagtgctaggagcggtgtcatgaacgccttcatcctgttgttgtgcgaggctcccttctgctgccagtttgtggagtttgcaaacacagtagctgagaaggttgaccggctgcgctcctggcaga

SEQ ID NO: 44 - protein, 73 aa, mus musculus
MSGSGAAGAAAGPAPPAQEEGMTWWYRWLCRLAGVLGAVSTPSSCCCARLPSAASLWSLQTQLRRLTGCAPGR